(19) United States
(12) Patent Application Publication
(10) Pub. No.: US 2009/0317461 A1
(43) Pub. Date: Dec. 24, 2009

(54) ORAL COMPOSITIONS EFFECTIVE FOR THE TREATMENT OF ORAL CAVITY MALODOR ASSOCIATED WITH THE CONSUMPTION OF ODOR-CAUSING COMPOUNDS

(75) Inventors: Russell A. Bazemore, Grant, AL (US); Charles J. Harrison, Ann Arbor, MI (US)

Correspondence Address: Derick E. Allen (28850);ARMSTRONG TEASDALE LLP, ONE METROPOLITAN SQUARE, SUITE 2600, ST. LOUIS, MO, 63102-2740, US

(73) Assignee: Wm. Wrigley Jr. Company, Chicago, IL (US)

(21) Appl. No.: 12/375,751

(22) PCT Filed: Jul. 27, 2007
(86) PCT No.: PCT/US2007/074624

§ 371 Date: Jun. 4, 2009

Related U.S. Application Data
(60) Provisional application No. 60/821,175, filed on Aug. 2, 2006.

(51) Int. Cl. A61K 9/20; A61K 9/68; A61K 8/35; A61K 8/97; A61P 1/02

(52) U.S. Cl. 424/464; 424/48; 424/49; 424/58

(57) ABSTRACT

This invention generally relates to oral compositions (e.g., a confection or chewing gum product) effective for the treatment of oral cavity malodor caused by odor-causing compounds (e.g., diallyl disulfide) associated with the consumption of, for example, garlic and/or onion. In particular, this invention relates to oral compositions containing menthone, isomenthone and, optionally, caryophyllene in varying concentrations, to provide an oral composition effective for the treatment of oral cavity malodor caused by odor-causing compounds associated with the consumption of, for example, garlic and onion.

### FIELD OF THE INVENTION

This invention generally relates to oral compositions (e.g., a confection or chewing gum product) effective for the treatment of oral cavity malodor caused by odor-causing compounds (e.g., diallyl disulfide) associated with the consumption of, for example, garlic and onion. In particular, this invention relates to oral compositions containing menthone and its stereoisomer, isomenthone and, optionally, caryophyllene in varying concentrations or proportions to provide an oral composition effective for the treatment of oral cavity malodor caused by odor-causing compounds associated with the consumption of, for example, garlic and onion.

### SUMMARY OF THE INVENTION

Briefly, therefore, the present invention is directed to oral compositions comprising menthone and its stereoisomer isomenthone and, optionally, caryophyllene that are effective for the reduction of oral malodor associated with the consumption of odor-causing compounds present in, for example, garlic and onions. In one embodiment, the composition comprises a source composition that contains menthone and isomenthone, the total concentration of menthone and isomenthone in the source composition being at least about 60% by weight. In another embodiment, the concentration of menthone is at least about 0.01% by weight, in particular, at least about 0.01% by weight of menthone derived from a source of menthone containing at least about 60% by weight menthone. In a still further embodiment, the oral composition comprises a flavorant source composition consisting essentially of menthone and isomenthone.

In various other embodiments, the present invention is directed to a chewing gum composition for reducing oral malodor associated with the consumption of odor-causing compounds present in, for example, garlic and/or onions. The chewing gum composition comprises (a) a water soluble bulk portion; (b) at least one flavoring agent; (c) a gum base portion; and (d) an effective amount of menthone, isomenthone, caryophyllene, or a combination thereof. The menthone, isomenthone, and/or caryophyllene is derived from a source composition having a concentration of menthone, isomenthone, or caryophyllene of at least 60% by weight.

The present invention is also directed to methods for the preparation of oral compositions effective for reducing oral malodor associated with the consumption of odor-causing compounds present in, for example, garlic and/or onions. In particular, the present invention is directed to such a method that comprises introducing a source composition containing menthone and isomenthone into a mixture of other components of the oral composition, the source composition having a concentration of menthone and isomenthone of at least about 60% by weight.

The present invention is further directed to oral compositions comprising menthone and caryophyllene effective for reducing oral malodor associated with the consumption of odor-causing compounds present in, for example, garlic and/or onions. In one embodiment, the composition comprises menthone and caryophyllene at a weight ratio of menthone to caryophyllene of less than 135:1. In this or another embodiment, the sum of the concentration of menthone and caryophyllene is at least 0.25% by weight. In this or yet a further embodiment, the menthone and caryophyllene are derived from a single source or oral composition flavor component. The present invention is also directed to methods for the preparation of an oral composition comprising adding a flavor component which contains both menthone and caryophyllene to the composition.

The present invention is further directed to an oral composition effective for reducing oral malodor which comprises a non-mint flavor and a source composition containing both menthone and isomenthone. The total concentration of menthone and isomenthone in the source composition is at least about 60% by weight.

In still further embodiments, the present invention is directed to an oral composition effective for reducing oral malodor that comprises (i) a source composition containing both menthone and isomenthone, and (ii) magnolia bark extract.

### BRIEF DESCRIPTION OF THE DRAWINGS

FIG. 1 shows the chemical structure of menthone.

FIG. 2 shows the chemical structure of caryophyllene, and more specifically trans-caryophyllene.

FIG. 3 is a total ion current (TIC) chromatogram prepared as described in Example 1.

FIG. 4 shows the percent reduction of diallyl disulfide headspace concentration observed with addition of menthone to a raw garlic solution at varying levels as described in Example 1.

FIG. 5 shows the percent reduction of diallyl disulfide headspace concentration observed with addition of caryophyllene to a raw garlic solution at varying levels as described in Example 1.

FIG. 6 shows sensory analysis results associated with treatment of a raw garlic solution with varying concentrations of menthone as described in Example 1.

FIG. 7 shows sensory analysis results associated with treatment of a raw garlic solution with varying concentrations of caryophyllene as described in Example 1.

FIG. 8 shows the rate of menthone release during 25 minutes of chewing in terms of the percentage of menthone released from the gum as described in Example 2.

FIG. 9 shows the rate of menthone release during 25 minutes of chewing in terms of the amount of menthone (μg) released from the gum as described in Example 2.

FIG. 10 shows the rate of caryophyllene release during 25 minutes of chewing in terms of the percentage of caryophyllene released from the gum as described in Example 2.

FIG. 11 shows the rate of caryophyllene release during 25 minutes of chewing in terms of the amount of caryophyllene (μg) released from the gum as described in Example 2.

FIG. 12 shows the percent reduction of diallyl disulfide headspace concentration observed with addition of menthone to a raw garlic solution as described in Example 3.

FIG. 13 shows the peak area of total ion current (TIC) chromatograms for various samples as described in Example 4.

FIG. 14 shows the peak area of total ion current (TIC) chromatograms for various samples as described in Example 4.

FIG. 15 shows the peak area of total ion current (TIC) chromatograms for various samples as described in Example 4.

FIG. 16 shows the diallyl disulfide headspace concentration observed with addition of menthone or menthone and caryophyllene to a raw garlic solution as described in Example 4.

FIG. 17 shows the peak area of total ion current (TIC) chromatograms for various samples described in Example 47.

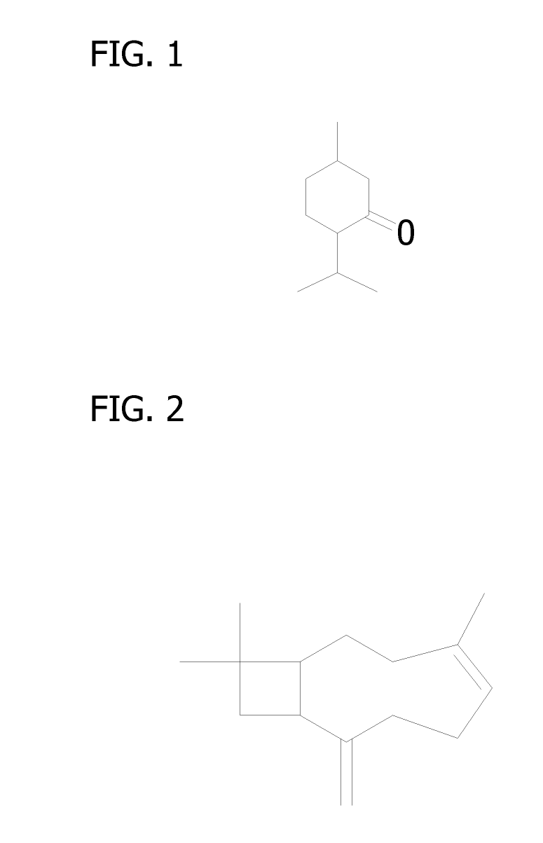

### DETAILED DESCRIPTION OF THE PREFERRED EMBODIMENTS

In accordance with the present invention, and as further detailed herein below, it has been discovered that oral compositions containing one or more of menthone, isomenthone, and caryophyllene are effective for treatment of oral cavity malodor associated with the consumption of foods derived from the plant genus Allium, such as garlic and onions. In particular, it has been discovered that oral compositions containing menthone and isomenthone are effective in reducing the concentration of volatile, odor-causing compounds present in a subject's oral cavity due to the consumption of such foods (e.g., garlic), and more specifically diallyl disulfide. It has further been discovered that oral compositions containing menthone and caryophyllene are likewise effective in reducing the concentration of volatile, odor-causing compounds present in a subject's oral cavity due to the consumption of such foods. In particular, it has been discovered that there may be a synergistic effect associated with inclusion of caryophyllene in an oral composition along with menthone. That is, the same total amount of menthone and caryophyllene may provide a greater reduction of volatile components associated with oral cavity malodor due to consumption of, for example, garlic or onion than an equal amount of menthone or caryophyllene alone.

Menthone and its stereoisomer, isomenthone, are typically present in flavoring agents such as peppermint oil and various other essential oils. Four distinct geometrical isomers of menthol are also often present as natural components of flavoring agents such as peppermint oil and various other essential oils. These isomers are menthol, neomenthol, isomenthol, and neoisomenthol. Each of these six compounds has an optical isomer, yielding a total of twelve compounds typically present in various flavoring agents. A particular compound of interest may be obtained by a variety of routine chemical manipulations, but purification of the compound of interest may be complicated by the co-synthesis of one or more isomers. As a result, additional separation techniques may be required to isolate individual compounds (e.g., derivitization, fractional crystallization, and/or spinning band distillation).

### I. Oral Compositions

Oral compositions in which menthone, isomenthone, and/or caryophyllene may be incorporated to provide a composition suitable for combating oral cavity malodor associated with the consumption of garlic include chewing gums, confections (e.g., lozenges and pressed tablets), edible films, mouthsprays, dentrifices, mouthwashes, foams, and toothpaste products.

A. Menthone, Isomenthone, Caryophyllene

In accordance with the present invention, menthone and isomenthone, alone or in combination, may be incorporated directly into the oral composition. The menthone and isomenthone are typically derived from sources other than flavor oils, including, for example, neat or substantially pure sources of menthone and isomenthone. In this regard it is to be noted that, as used herein, reference to a neat or a substantially pure source of menthone and isomenthone refers to a source composition having a total concentration of menthone and isomenthone of at least about 60% by weight or higher. Typically, however, the total menthone and isomenthone concentration in the source composition is at least about 70% by weight, at least about 80% by weight, at least about 90% by weight, at least about 95% by weight, or even at least about 99% by weight. In various embodiments, such a neat or substantially pure menthone/isomenthone source may be mixed with another flavor source and added to the oral composition (e.g., a chewing gum). In addition, in various embodiments, a portion of the menthone and/or isomenthone present in the oral composition may optionally be derived from flavor oils generally known in the art including, for example, peppermint oil and spearmint oil.

Menthone and isomenthone, existing as stereoisomers in many essential oils and substantially pure sources of menthone and isomenthone, have a strong tendency for interconversion. Thus, most sources of menthone are mixtures of menthone and isomenthone in varying proportions. Because of this tendency for interconversion, menthone/isomenthone sources incorporated into oral compositions of the present invention typically contain menthone and isomenthone at a weight ratio of at least about 50:50, or at least about 60:40, and may in some embodiments have a weight ratio of menthone to isomenthone of at least about 70:30, at least about 80:20, at least about 90:10, or even at least about 95:5. Accordingly, in various embodiments the weight ratio of menthone and isomenthone in the oral composition flavor component or source composition may be from about 50:50 to about 95:5, or from about 70:30 to about 90:10, or in particular about 80:20. For example, a commercially available 80:20 (weight:weight) mixture of menthone to isomenthone suitable for use in oral compositions of the present invention is available from I.P. Callison and Sons (2400 Callison Road NE, Lacey, Wash. 9851).

Similarly, caryophyllene (particularly β-caryophyllene) may be incorporated directly into the oral composition (e.g., by virtue of a neat or a substantially pure source of caryophyllene) or derived from a source of caryophyllene. As used herein, reference to a neat or substantially pure source of caryophyllene refers to a source that is not an essential oil and/or a source that contains a relatively high proportion of caryophyllene. Generally speaking, these sources contain at least about 60% by weight, at least about 70% by weight, at least about 80% by weight, at least about 90% by weight, at least about 95% by weight, or even at least about 99% by weight caryophyllene. Substantially pure caryophyllene is commercially available from, for example, Citrus and Allied Essences LTD (3000 Marcus Avenue, Lake Success, N.Y. 11042).

It should be understood that at least a portion of the caryophyllene present in the oral composition may be derived from other sources that are not necessarily classified as “neat” sources. Caryophyllene is a sesquiterpenoid occurring in many essential oils such as clove oil, as well as oils derived from various stems and flowers. Sources of caryophyllene are well-known in the art and include a variety of flavor oils, including but not limited to, citrus, black currant, capsicum sp., ginger, grape, guava, juniper, mango, rosemary, raspberry, tea, clove, lavender, hop, spearmint, bergamot, oregano, curry, basil, and angelica.

Typically, menthone and/or its stereoisomer isomenthone are present in oral compositions of the present invention at a concentration of at least about 0.0001%, at least about 0.001%, at least about 0.01%, at least about 0.1%, by weight, or more (e.g., at least about 0.2%, at least about 0.3%, at least about 0.4%, or even at least about 0.5%, by weight) and/or less than about 20%, less than about 10%, less than about 5%, or even less than 1% by weight. For example, in some embodiments of the present invention menthone and/or isomenthone may be present in an oral composition at a concentration of from about 0.001% to about 20% by weight, from about 0.001% to about 10% by weight, from about 0.01% to about 5% by weight, or from about 0.1% to about 1% by weight. Alternatively, menthone and/or isomenthone may be present in an oral composition at a concentration of from about 0.1% to about 1% by weight, from about 0.2% to about 0.8%, or from about 0.3% to about 0.6% by weight of the composition.

It is to be noted that, in these or other various embodiments, caryophyllene may be present in the oral composition along with menthone and/or isomenthone. For example, caryophyllene may be present in such embodiments at a concentration of at least about 0.0001%, at least about 0.001%, at least about 0.01%, at least about 0.1%, and/or less than about 20%, less than about 10%, less than about 5%, or even less than about 1% by weight. For example, caryophyllene may be present in an oral composition at a concentration of from about 0.001% to about 20% by weight, from about 0.001% to about 10% by weight, from about 0.01% to about 5% by weight, or from about 0.1% to about 1% by weight. In embodiments in which the oral composition comprises both menthone (and/or isomenthone) and caryophyllene, menthone/isomenthone and caryophyllene may be provided by a single source (e.g., erospicata oil or spearmint oil). For example, in one embodiment, erospicata oil, which is known to contain approximately 50-55% by weight menthone and approximately 2.5-3.5% by weight caryophyllene, may be incorporated into the oral composition. In particular, erospicata oil may be present in the oral composition at concentrations of up to about 30% by weight (e.g., about 5%, about 10%, about 15%, about 20%, about 25% by weight, or more).

It is to be still further noted that, in one or more of the above-noted embodiments, or alternatively in yet another embodiment, the sum of the concentrations of menthone and isomenthone mixtures and caryophyllene, and relative amounts thereof, may be at certain optimum levels and within certain optimum ranges, depending upon the desired characteristics of the oral composition. Typically, however, the sum of the concentration of menthone and caryophyllene is at least 0.25% by weight, at least about 0.35% by weight, at least about 0.45% by weight or, even more typically, at least about 0.55% by weight of the composition. For example, the sum of the concentration of menthone and caryophyllene may be from at least 0.25% to about 1.5% by weight, from about 0.4% to about 1% by weight, or from about 0.5% to about 0.8% by weight.

It is to be still further noted that, in one or more of the above-noted embodiments, or alternatively in yet another embodiment, the weight ratio of menthone (or the combination of menthone and isomenthone) to caryophyllene may typically be less than about 135:1, less than about 100:1, less than about 75:1, less than about 50:1, or even less than about 25:1. In these and/or various other embodiments, the weight ratio of menthone (or the combination or menthone and isomenthone) to caryophyllene may be at least about 0.5:1, at least about 1:1, or least about 1.5:1. For example, in various embodiments, the weight ratio of menthone (or the combination or menthone and isomenthone) to caryophyllene may be from about 0.5:1 to about 10:1, about 0.75:1 to about 9:1 or about 1:1 to about 5:1 (e.g., about 2.5:1 or about 1:1).

### II. Chewing Gums

In various embodiments of the present invention, the oral composition is in the form of a chewing gum. A chewing gum composition generally comprises a water-soluble bulk portion, a water-insoluble chewable gum base portion, and one or more water-soluble flavoring agents. The water-soluble bulk portion dissipates with a portion of the flavoring agent(s) over a period of time during chewing while the gum base portion is retained in the mouth throughout the chew.

Flavoring agents that provide the desired flavors for chewing gums and other oral compositions of the present invention may include both mint-derived and/or non-mint flavors generally known to those skilled in the art, including essential oils, synthetic flavors, and mixtures thereof. Such flavoring agents include, but are not limited to, oils derived from plants and fruits such as citrus oils, fruit essences, mint oils, and non-mint oils (e.g., clove oil, anise and the like). Fruit flavors may in particular include lemon, orange, lime, grapefruit, tangerine, strawberry, apple, cherry, raspberry, blackberry, blueberry, banana, pineapple, cantaloupe, muskmelon, watermelon, grape, currant, mango, kiwi and many others as well as combinations. Mint flavors may in particular include spearmint, peppermint, wintergreen, basil, corn mint, menthol and others and mixtures thereof. In addition to the fruit flavors noted above, non-mint flavors may include spice flavors such as, for example, cinnamon, vanilla, clove, chocolate, nutmeg, coffee, licorice, eucalyptus, ginger, cardamom and many others. Other non-mint flavors may include herbal and savory flavors such as popcorn, chili, corn chip and the like. Artificial flavoring agents and components may also be used. Natural and artificial flavoring agents may be combined in any sensorial acceptable fashion.

Flavoring may also include a cooling agent to enhance the flavor and perceived breath freshening of the product. Cooling agents are trigeminal stimulants that impart a cool sensation to the mouth, throat, and nasal passages. The most common cooling agent is menthol, although it may typically be considered as a flavor due to its aroma properties and the fact that it is a natural component of peppermint oil. Typically, cooling agents include other natural or synthetic chemicals used to impart a cooling sensation with minimal aroma. Commonly employed cooling agents include, for example, menthol, ethyl p-menthane carboxamide and other N-substituted p-menthane carboxamides, N,2, 3-trimethyl-2-isopropyl-butanamide and other acyclic carboxamides, menthyl glutarate (Flavor Extract Manufacturing Association (FEMA 4006)), 3-1-menthoxypropane-1,2-diol, isopulegol, menthyl succinate, menthol propylene glycol carbonate, menthol ethylene glycol carbonate, menthyl lactate, menthyl glutarate, menthone glyceryl ketal, p-menthane-1,8-diol, menthol glyceryl ether, N-tertbutyl-p-menthane-3-carboxamide, p-menthane-3-carboxylic acid glycerol ester, methyl-2-isopryl-bicyclo(2.2.1), heptane-2-carboxamide, menthol methyl ether, and combinations thereof. Trigeminal stimulants other than cooling agents may also be employed. These include warming agents such as capsaicin, capsicum oleoresin, red pepper oleoresin, black pepper oleoresin, piperine, ginger oleoresin, gingerol, shoagol, cinnamon oleoresin, cassia oleoresin, cinnamic aldehyde, eugenol, cyclic acetal of vanillin, menthol glycerin ether and unsaturated amides and tingling agents such as Jambu extract, Vanillyl alkyl ethers such as Vanillyl n-butyl ether, sanshool, spilanthol, Echinacea extract and Northern Prickly Ash extract. These components may also be used as flavoring agents.

Any of these flavoring agents (either alone or in combination with a cooling agent) may be present in amounts of from about 0.1% to about 15% by weight of the chewing gum and, more typically, in amounts from about 0.2% to about 5% by weight of the chewing gum.

In various embodiments, the effective amount of the active (i.e., menthone, isomenthone, or caryophyllene) may be present in the coating of a chewing gum composition, while in others all or a portion of the effective amount may be encapsulated within the chewing gum composition.

Menthone, isomenthone and/or caryophyllene may be encapsulated alone or in any combination to increase or delay their release from the oral composition by modifying the solubility or dissolution rate. Any technique known to those skilled in the art which gives partial or full encapsulation of the flavoring agents(s) can be used. These techniques include, but are not limited to, spray drying, spray chilling, fluid-bed coating and coacervation (U.S. Pat. No. 3,712,867 describes coacervation as an encapsulation technique). These encapsulation techniques can be used individually or in any combination in a single step process or multiple step process. Generally, delayed release of the breath freshening compounds can be obtained using multistep processes that include spray drying the breath freshening compound and fluid-bed coating of the resultant powder.

The encapsulation techniques of the breath freshening compounds here described are standard coating techniques that generally give varying degrees of coating, from partial to full coating, depending on the coating composition (e.g., encapsulant) used in the process. Coating compositions may be susceptible to water permeation to various degrees. Generally, compositions that have high organic solubility, good film-forming properties and low water solubility exhibit delayed release of the breath freshening compounds. These include acrylic polymers and copolymers, carboxyvinyl polymer, polyamides, polystyrene, polyvinyl acetate, polyvinyl acetate phthalate, polyvinylpyrrolidone, and waxes. Although all of these materials are possible for encapsulation of the breath freshening compounds, two standard food-grade coating materials that are good film formers but not water soluble are shellac and Zein. Others which are more water soluble, but good film formers, are materials like agar, alginates, a wide range of cellulose derivatives like ethyl cellulose, methyl cellulose, sodium hydroxymethyl cellulose, and hydroxypropylmethyl cellulose, dextrin, gelatin, and modified starches. Other encapsulants like acacia or maltodextrin may also be used to encapsulate the breath freshening compounds, and provide the desired release from various oral compositions.

The insoluble gum base generally comprises elastomers, resins, fats and oils, softeners, and inorganic fillers and may also include wax. The insoluble gum base may constitute approximately 5% to about 95% by weight of the chewing gum. However, the gum base typically constitutes from about 10% to about 50% of the chewing gum and, more typically, from about 25% to about 35% by weight of the chewing gum.

In various embodiments, the chewing gum base contains from about 20% to about 60% by weight of a synthetic elastomer, up to about 30% by weight of a natural elastomer, from about 5% to about 55% by weight of an elastomer plasticizer, from about 4% to about 35% by weight of a filler, from about 5% to about 35% by weight of a softener, and optional minor amounts (e.g., about 1% or less by weight) of miscellaneous ingredients such as colorants, antioxidants, etc.

Synthetic elastomers may include, but are not limited to, polyisobutylene having a GPC weight average molecular weight of about 10,000 to about 95,000, isobutylene-isoprene copolymer (butyl elastomer), styrene-butadiene copolymers (having styrene-butadiene ratios of, for example, about 1:3 to about 3:1), polyvinyl acetate having GPC weight average molecular weight of about 2,000 to about 90,000, polyisoprene, polyethylene, vinyl acetate-vinyl laurate copolymer having vinyl laurate content of about 5% to about 50% by weight of the copolymer, and combinations thereof.

Preferred synthetic elastomers include polyisobutylene having a GPC weight average molecular weight of from about 50,000 to 80,000, styrene-butadiene copolymers having a styrene-butadiene ratio for bound styrene of from 1:1 to 1:3, polyvinyl acetate having a GPC weight average molecular weight of from 10,000 to 65,000, with the higher molecular weight polyvinyl acetates typically used in bubble gum base, and vinyl acetate-vinyl laurate copolymer having a vinyl laurate content of 10.

Natural elastomers may include natural rubber, such as smoked or liquid latex and guayule, as well as natural gums, such as jelutong, lechi caspi, perillo, sorva, massaranduba balata, massaranduba chocolate, nispero, rosindinha, chicle, gutta hang kang, and combinations thereof. The preferred synthetic elastomer and natural elastomer concentrations vary depending on whether the chewing gum in which the base is used is adhesive or conventional, bubble gum or regular gum. Preferred natural elastomers include jelutong, chicle, sorva, and massaranduba balata.

Elastomer plasticizers may include, but are not limited to, natural rosin esters such as glycerol esters or partially hydrogenated rosin, glycerol esters of polymerized rosin, glycerol esters of partially dimerized rosin, glycerol esters of rosin, pentaerythritol esters of partially hydrogenated rosin, methyl and partially hydrogenated methyl esters of rosin, pentaerythritol esters of rosin; synthetics such as terpene resins derived from alpha, beta, and/or any suitable combinations of the foregoing. The preferred elastomer plasticizers will also vary depending on the specific application, and on the type of elastomer which is used.

The gum typically also includes a filler component. Fillers and/or texturizers may include inorganic powders such as magnesium and calcium carbonate, ground limestone, silicate types such as magnesium and aluminum silicate, clay, alumina, talc, titanium oxide, mono-, di- and tri-phosphate, cellulose polymers, such as wood, and combinations thereof.

Softeners and/or emulsifiers may include tallow, hydrogenated tallow, hydrogenated and partially hydrogenated vegetable oils, cocoa butter, glycerol monostearate, glycerol triacetate, lecithin, mono and triglycerides, acetylated monoglycerides, fatty acids (e.g. stearic, palmitic, oleic and linoleic acids), and combinations thereof.

Colorants and whiteners may include FD&C dyes and lakes, fruit and vegetable extracts, titanium dioxide, and combinations thereof.

As noted elsewhere herein, the gum base may include wax. However, an example of a wax-free gum base is disclosed in U.S. Pat. No. 5,286,500, the disclosure of which is incorporated herein by reference.

In addition to a water insoluble gum base portion, a typical chewing gum composition further includes a water soluble bulk portion. The water soluble portion can include bulk sweeteners, high intensity sweeteners, flavoring agents, softeners, emulsifiers, colors, acidulants, fillers, antioxidants, and other components that provide desired attributes.

Softeners are added to the chewing gum in order to optimize the chewability and mouthfeel of the gum. The softeners, which are also known as plasticizers and plasticizing agents, generally constitute from about 0.5% to about 15% by weight of the chewing gum. The softeners may include glycerin, lecithin, and combinations thereof. Aqueous sweetener solutions such as those containing sorbitol, hydrogenated starch hydrolysates (e.g., hydrogenated starch hydrolysate syrups or maltitol syrups), corn syrup, and combinations thereof may also be used as softeners and binding agents in the chewing gum. Aqueous softeners may be combined with glycerin or propylene glycol to produce co-evaporated syrups such as those described, for example, in U.S. Pat. No. 4,671,961. An emulsifier may be incorporated to improve the consistency and stability of the gum product, and also contribute to product softness. Lecithin is the most commonly employed emulsifier, although nonionic emulsifiers such as polyoxyethylene sorbitan fatty acid esters and partial esters of common fatty acids (lauric, palmitic, stearic and oleic acid hexitol anhydrides (hexitans and hexides) derived from sorbitol may also be used. When used, emulsifiers typically comprised 0.5 to 2% of the chewing gum composition.

Surface active agents may also be incorporated into chewing gums of the present invention. These include, for example, salts of potassium, ammonium, or sodium. Sodium salts include anionic surface active agents, such as alkyl sulfates, including sodium lauryl sulfate, sodium laureth sulfate, and the like. Other sodium salts include sodium lauroyl sarcosinate, sodium brasslate, and the like. Suitable ammonium salts include betaine derivatives such as cocamidopropyl betaine, and the like.

Moisture may be added to the chewing gums as a separate ingredient, but is more typically a minor component by virtue of the moisture contents of other ingredients. While almost all food ingredients contain some water, most of the water is contributed by carbohydrate syrups where present. Other components which may contribute significant amounts of moisture include certain bulking agents, glycerin and occasionally other ingredients. The total amount of moisture in a chewing gum product impacts its texture and stability and, if the product is not sufficiently protected by packaging, undesired moisture loss may occur. Initial moisture levels in chewing gums may be as little as 0.1%, by weight, or even less, or as high as 3 to 4%, by weight, depending on the type of gum, the ingredients used, the intended geographic market, the presence of moisture sensitive ingredients and other factors. Pellet centers typically exhibit relatively low moisture levels, while sugar stick gums often exhibit relatively high moisture levels.

Bulk sweeteners, or bulking agents, include both sugar and sugarless components. Bulk sweeteners typically constitute from about 5% to about 95% by weight of the chewing gum, more typically from about 20% to about 80% by weight of the chewing gum and, more typically, from about 30% to about 60% by weight of the gum. Sugar sweeteners generally include saccharide components commonly known in the chewing gum art, including but not limited to, sucrose, dextrose, maltose, dextrin, dried invert sugar, fructose, levulose, galactose, corn syrup solids, and the like, alone or in combination. Sugarless sweeteners include, but are not limited to, sugar alcohols such as sorbitol, mannitol, xylitol, maltitol, hydrogenated starch hydrolysates, erythritol, tagatose, trehalose, and the like, alone or in combination.

High intensity artificial sweeteners can also be used, alone or in combination, with the above. Preferred sweeteners include, but are not limited to, sucralose, aspartame, NAPM derivatives such as neotame, salts of acesulfame, altitame, saccharin and its salts, cyclamic acid and its salts, glycyrrhizin, dihydrochalcones, thaumatin, monellin, and the like, alone or in combination. In order to provide longer lasting sweetness and flavor perception, it may be desirable to encapsulate or otherwise control the release of at least a portion of the artificial sweetener. Such techniques as wet granulation, wax granulation, spray drying, spray chilling, fluid bed coating, coacervation, and fiber extension may be used to achieve the desired release characteristics.

Combinations of sugar and/or sugarless sweeteners may be used in the chewing gum. Additionally, the softener may also provide additional sweetness such as with aqueous sugar or alditol solutions.

If a low calorie gum is desired, a low calorie bulking agent can be used. Examples of low caloric bulking agents include polydextrose, raftilose, raftilin, fructooligosaccharides (e.g., NutraFlora®), Palatinose oligosaccharide, guar gum hydrolysate (e.g., Sun Fiber®), or indigestible dextrin (e.g., Fibersol®).

Optionally, the chewing gum of the present invention may include additional breath freshening, anti-microbial or oral health ingredients, such as food acceptable metallic salts selected from zinc and copper salts of gluconic acid, zinc and copper salts of lactic acid, zinc and copper salts of acetic acid, zinc and copper salts of citric acid, copper chlorophyll and combinations thereof. Chewing gums of the present invention may also include one or more food acids (e.g., ascorbic acid) that typically provide a tart, or sour, flavor to fruit-flavored products. The nature and release of tartness may be controlled by selection of the particular food acid, and its concentration in the product.

Chewing gum generally conveys oral care benefits. In addition to mechanical cleaning of the teeth provided by the chewing action, saliva stimulated by chewing, flavor and taste from the product conveys beneficial properties in reducing bad breath, neutralizing acid, and remineralizing teeth. Saliva also contains beneficial polypeptides and other components which may improve the oral environment. These include: antimicrobial proteins, such as lysozyme, lactoferrin, peroxidases, and histatins; inhibitors of spontaneous crystallization, such as statherin. To assist in providing these benefits, chewing gums of the present invention may be used as vehicles for the delivery of specialized oral care agents. These may include antimicrobial compounds such as Cetylpyridinium Chloride (CPC), triclosan, chlorhexidine, and magnolia bark extract (MBE); anti-caries agents such as calcium and phosphate ions, plaque removal agents such as abrasives, surfactants and enzymes; plaque neutralization agents such as ammonium salts, urea and other amines; anti-tartar/calculus agents such as soluble pyrophosphates salts; anti halitosis agents such as parsley oil and copper or zinc salts of gluconic acid, lactic acid, acetic acid or citric acid, and whitening agents such as peroxides; agents that may provide either local or systemic anti-inflammatory effects to limit gingivitis, such as COX-2 inhibitors; agents that may reduce dentinal hypersensitivity, such as potassium salts to inhibit nerve cell transmission, and calcium phosphate salts to block the dentinal tubules.

For example, the chewing gum may include anti-microbial essential oils and components such as peppermint, methyl salicylate, thymol, eucalyptol, cinnamic aldehyde, polyphosphate, pyrophosphate, clove oil (eugenol), and combinations thereof. Dental health ingredients, such as fluoride salts, phosphate salts, proteolytic enzymes, lipids, anti-microbials, calcium salts, electrolytes, protein additives, dental abrasives, and combinations thereof, may also be added to the gum composition.

By way of further example, oral compositions of the present invention (e.g., chewing gums), may include magnolia bark extract to desirably provide antimicrobial benefits, in addition to breath freshening benefits. It is currently believed that magnolia bark extract may provide suitable efficacy for either or both these purposes at relatively low levels. Depending on a variety of factors (e.g., purity of the source of MBE, the type of oral bacteria present, and the concentration of the oral bacteria), it is currently believed that MBE may eliminate a substantial proportion, if not substantially all, oral bacteria present in the oral cavity of a consumer.

As used herein, the term “magnolia bark extract” refers to an extract from dried cortex, or bark, of a plant from the Magnoliaceae family (such as Magnolia officinalis, and also refers to a synthetic or semi-synthetic equivalent of such an extract or one or more active compounds thereof. Components of MBE believed to contribute to its activity (e.g., oral care efficacy) include, for example, magnolol, honokiol, tetrahydromagnolol and tetrahydrohonokiol.

Reference to a neat or substantially pure source of MBE herein refers to a source that contains a relatively high proportion of any of the active components of the MBE. Generally speaking, these sources contain at least about 50% by weight, at least about 60% by weight, at least about 70% by weight, at least about 80% by weight, at least, or even at least about 90% by weight magnolol, honokiol, tetrahydromagnolol and tetrahydrohonokiol, either individually or in any combination.

Typically, magnolia bark extract may be present in oral compositions at a concentration of from about 0.001 to about 10% by weight, more typically from about 0.001 to about 8% by weight and, still more typically, from about 0.001 to about 6% by weight (e.g., from about 0.001 to about 4% by weight, from about 0.001 to about 2.5% by weight, or from about 0.001 to about 1% by weight). As noted, it is currently believed that MBE may provide suitable efficacy for antimicrobial and/or oral care benefits when present in oral compositions at relatively low concentrations. Thus, in various embodiments, MBE may be present in the oral composition at concentrations of less than about 0.8% by weight, less than about 0.5% by weight, or even less than about 0.1% by weight (e.g., about 0.08% by weight, or about 0.04% by weight).

It is to be noted that magnolia bark extract may be present in oral compositions along with menthone, isomenthone, and/or caryophyllene in the proportions detailed elsewhere herein. In particular, magnolia bark extract may be present in oral compositions along with neat, or substantially pure sources of menthone, isomenthone, and/or caryophyllene.

For example, in various embodiments, regardless of the precise composition of the source of menthone and/or the composition of the magnolia bark extract, menthone and magnolia bark extract may generally be present in the oral composition at a weight ratio of menthone to MBE of at least about 0.5:1, at least about 0.75:1, at least about 1:1, at least about 1.25:1, or at least about 1.5:1. For example, the weight ratio of menthone to MBE may be from about 0.5:1 to about 5:1, from about 0.75:1 to about 4:1, from about 1:1 to about 3:1, or from about 1:1 to about 2:1.

It has been observed that the combination of menthone and magnolia bark extract may be well-suited for incorporation into tabletted, or pelleted chewing gums that include a center and a coating. Typically in such embodiments, menthone is incorporated into the center of the gum, while magnolia bark extract is incorporated into the coating. In particular, desirably all, or substantially all of the menthone and magnolia bark extract (e.g., greater than about 90%, greater than about 95%, or greater than about 99% by weight) is present in the gum center and coating, respectively.

In general, the chewing gum is manufactured by sequentially adding the various chewing gum ingredients to a commercially available mixer known in the art. After the ingredients have been thoroughly mixed, the gum mass is discharged from the mixer and shaped into the desired form such as rolling sheets and cutting into sticks, extruding into chunks or casting into pellets, which are then coated or panned.

Generally, the ingredients are mixed by first melting the gum base and adding it to the running mixer; the base may also be melted in the mixer itself. Color or emulsifiers may also be added at this time. A softener such as glycerin may also be added at this time, along with syrup and a portion of the bulking agent. Further parts of the bulking agent are added to the mixer. Flavoring agents are typically added with the final portion of the bulking agent. Other optional ingredients are added to the batch in a typical fashion, well known to those of ordinary skill in the art.

Chewing gums of the present invention may be prepared in accordance with continuous mixing processes generally known in the art including, for example, those described in U.S. Pat. Nos. 5,543,160, 5,419,919 and 6,017,565, the entire contents of which are hereby incorporated by reference for all relevant purposes.

The entire mixing procedure typically takes from five to fifteen minutes, but longer mixing times may sometimes be required. Those skilled in the art will recognize that many variations of the above described procedure may be followed.

Chewing gum base and chewing gum product have been manufactured conventionally using separate mixers, different mixing technologies and, often, at different factories. One reason for this is that the optimum conditions for manufacturing gum base, and for manufacturing chewing gum from gum base and other ingredients such as sweeteners and flavors, are so different that it has been impractical to integrate both tasks. Chewing gum base manufacture, on the one hand, involves the dispersive (often high shear) mixing of difficult-to-mixture ingredients such as elastomer, filler, elastomer plasticizer, base softeners/emulsifiers and sometimes wax, and typically requires long mixing times. Chewing gum product manufacture, on the other hand, involves combining the gum base with more delicate ingredients such as product softeners, bulk sweeteners, high intensity sweeteners and flavoring agents using distributive (generally lower shear) mixing, for shorter periods.

Examples of chewing gum formulations containing a menthone/isomenthone mixture and, optionally, caryophyllene in accordance with the present invention are set forth below in Examples 5-20.

The chewing gums of the present invention may be coated or uncoated. The chewing gum coatings may be incorporated using methods generally known in the art including, for example, pan coating. Examples of coatings and coated chewing gums containing menthone/isomenthone mixtures and caryophyllene are set forth below in Examples 21-29.

### III. Confections

In various embodiments of the present invention, the oral composition is in the form of a confection such as a hard candy, chewy candy, coated chewy center candy, toffee, syrup, nougat, chocolate and tableted candy. Generally, menthone and, optionally, caryophyllene are present in the confection in the overall and relative proportions and provided by the sources detailed elsewhere herein regarding oral compositions generally. In various embodiments, however, menthone and/or caryophyllene are present in the confection at a concentration of from about 0.001% to about 4% or from about 0.001% to about 1% by weight of the confection. In various other embodiments, menthone and/or caryophyllene are present in the confection at a concentration of about 0.01% or about 1% by weight. In still other embodiments, higher concentrations of menthone and/or caryophyllene (e.g., from about 5% to about 15% by weight) may be desired.

A. Hard Candy

A hard candy is primarily comprised of corn syrup and sugar, and derives its name from the fact that it typically contains from about 1% to about 4% moisture. In appearance, these types of candies are solid, but they are actually super cooled liquids existing at far below their melting points. There are different types of hard candies. Glass types are usually clear or made opaque with dyes. Grained types of hard candies are always opaque. Examples of hard candy formulations are set forth below in Examples 30-35.

One method for manufacture of a glass type hard candy having a sugar base involves spreading the corn syrup over a cylinder heated by high pressure steam. Rapid heat exchange causes the water in the syrup to evaporate. The cooked syrup is discharged and colors and flavors are added. The syrup is cooled and deposited onto a stainless steel conveyor. The syrup can be conveyed directly to hoppers which then discharge directly into molds. The candy is conveyed to batch rollers, which shapes and sizes the batch. The candy enters a former, which shapes the individual pieces into discs, balls, barrels, etc. Hard candies of the present invention can be made into any shape, circles, squares, triangles, etc., also into animal shapes or any other novelty molding available. The candy is then cooled, wrapped and packaged.

For grained types of candy, water and sugar are the basic components that are mixed with other ingredients (e.g., flavoring agents). The ingredients are cooked at high temperatures (i.e., from approximately 143°-155° C. (approximately 290-310° F.)), causing the water to turn to steam. The product is transferred to a cooling wheel, where it is collected in about 68 kg (approximately 150 pound) batches, placed in a pulling machine to aerate the product, and the flavor is added. The candy is transferred to batch rollers where it is shaped and sized. The candy then enters a former, which shapes the individual pieces. The candy is cooled at a relative humidity of 35% and enters a rotating drum where it is coated with a fine sugar. The candy is then conveyed to the graining room for four hours at approximately 32° C. (90° F.) and 60% humidity. The entrapped air and moisture causes the product to grain.

B. Pressed Tablets and Lozenges

In various other embodiments, the oral composition is in the form of a pressed tablet (e.g., pressed mint) or lozenge (e.g., a boiled drop) manufactured using conventional techniques. Preferably, in various embodiments, these tablets or lozenges are slow dissolving. Generally, pressed tablets and lozenges are prepared in the same manner with the same general formula; however, lozenge formulations typically contain up to about 2% by weight of a hydrocolloid as a barrier agent to provide a shiny surface while pressed tablets do not contain a barrier agent and typically have a smooth finish. Typical pressed mint formulations are set forth below in Examples 36, and 38-42.

It is currently believed that the combination of menthone and magnolia bark extract may be well-suited for incorporation into pressed tablets. In various embodiments, these pressed tablets may be multi-layered. Typically in such embodiments, menthone may be incorporated into one layer of the tablet, while magnolia bark extract may be incorporated into another, different layer. In particular, desirably all, or substantially all of the menthone (e.g., greater than about 90%, greater than about 95%, or greater than about 99% by weight) is present in one layer of the tablet, and all, or substantially all (e.g., greater than about 90%, greater than about 95%, or greater than about 99% by weight) of the magnolia bark extract is present in a different layer.

The tablets and lozenges generally include one or more flavorants. These flavorants may provide the desired active (e.g., menthone, caryophyllene) content while the tablet or lozenge may also include additional active (e.g., substantially pure menthone). Suitable flavorants include natural and artificial flavors and mints, such as oil of peppermint, menthol, oil of spearmint, vanilla, oil of cinnamon, oil of wintergreen (methyl salicylate), and various fruit flavors, including but not limited to lemon oil, orange oil, grape flavor, lime oil, grapefruit oil, apple, apricot essence, and combinations thereof. Without limiting the foregoing discussion concerning menthone, caryophyllene, and menthol contents of the oral compositions of the present invention, the flavorings are generally utilized in amounts that will vary depending upon the individual flavor, and may, for example, range in amounts of about 0.5% to about 3% by weight of the tablet or lozenge.

Pressed tablets and lozenges of the present invention typically comprise a solid carrier in the form of a sugar or a water soluble polyhydric alcohol (polyol) such as mannitol, xylitol, sorbitol, maltitol, a hydrogenated starch hydrolysate (e.g., “Lycasin”), hydrogenated glucose, hydrogenated disaccharides, and/or hydrogenated polysaccharides, as the major ingredient, in an amount of from about 85 to about 98% by weight of the total carrier. Solid salts such as sodium bicarbonate, sodium chloride, potassium bicarbonate or potassium chloride may totally or partially replace the polyol carrier.

The pressed tablet or lozenge may further comprise tableting lubricants in minor amounts (e.g., from about 0.1% to about 5% by weight) to facilitate the preparation of both the tablets and the lozenges. Suitable lubricants include vegetable oils (e.g., coconut oil), calcium stearate, magnesium stearate, amino acids, aluminum stearate, talc, starch, and Carbowax.

Tablets and lozenges of the present invention may optionally include a coating material such as waxes, shellacs, carboxymethyl cellulose, polyethylene/maleic anhydride copolymer or Kappa-carrageenan, to further increase the time it takes the tablet or lozenge to dissolve in the mouth. In various embodiments, a slow dissolving tablet or lozenge is preferred in order to provide a sustained release rate of the active ingredients over a period of time of from about 3 to about 15 minutes.

Tablets and lozenges of the present embodiment typically also include optional sweeteners and or colorants.

Sweeteners may be one or more sweeteners known in the art, including both natural and artificial sweeteners. The sweetener may be chosen from a wide range of materials, including water-soluble sweeteners, water-soluble artificial sweeteners, and dipeptide based sweeteners and mixtures thereof. Thus, sweeteners may be chosen from the following non-limiting list, which includes sugars such as sucrose, glucose, corn syrup, dextrose, invert sugar, fructose, and mixtures thereof; saccharine and its various salts such as the sodium or calcium salt; cyclamic acid and its various salts such as the sodium salt; free aspartame; dihydrochalcone sweetening compounds; glycyrrhizin; stevioside; monellin, thaumatin, sucralose, isomaltitol, neotame, lactitol, trehalose, lactosucrose, polydextrose, tagatose, perillartine; and sugar alcohols such as sorbitol, sorbitol syrup, mannitol, maltitol, erythritol, xylitol, and the like. Also contemplated as a sweetener is the nonfermentable sugar substitute hydrogenated starch hydrolysate (also known as Lycasin). Also contemplated is the synthetic sweetener 3,6-dihydro-6-methyl-1-1,2,3-oxathiazin-4-one-2,2-dioxide, particularly the potassium (Acesulfame-K), sodium and calcium salts thereof. In various preferred embodiments, sorbitol is the sweetening and bulking agent. The amount of sweetener included is an amount effective to provide the desired degree of sweetness and bulk, generally from about 0.001% to about 70% by weight of the tablet or lozenge.

High intensity artificial sweeteners can also be used, alone or in combination, with other sweeteners. Preferred high intensity sweeteners include, but are not limited to, sucralose, aspartame, NAPM derivatives such as neotame, salts of acesulfame, alitame, stevia, saccharin and its salts, cyclamic acid and its salts, glycyrrhizinate, dihydrochalcones, thaumatin, monellin, and the like, alone or in combination. In order to provide longer lasting sweetness and flavor perception, it may be desirable to encapsulate or otherwise control the release of at least a portion of the artificial sweetener. Such techniques as wet granulation, wax granulation, spray drying, spray chilling, fluid bed coating, coacervation, and fiber extension may be used to achieve the desired release characteristics.

Colorants can be present in the tablets or lozenges of the present invention. Examples include pigments such as titanium dioxide, natural food colorants such as beta carotenes, betanin, turmeric, and other dyes suitable for food, drug and cosmetic applications known as F.D. & C. dyes, and the like. The materials may be incorporated in amounts of up to about 1% by weight, preferably up to about 6% by weight of the tablet or lozenge.

Tablets and lozenges of the present invention are generally prepared in accordance with conventional mixing and tableting techniques known in the art. For example, the pressed tablet into which the menthone and, optionally, caryophyllene are incorporated may be prepared by wet granulation, dry granulation, and direct compression methods. In general, wet granulation involves mixing milled powders, preparing a wet mass by blending the milled powders with a binder solution, coarse screening the wet mass and drying the moist granules, screening the granules through a 14 to 20 mesh screen, mixing the screened granules with lubricants and disintegrate agents and finally tablet compressing the mass. In contrast, dry granulation generally involves milling of powders, compression into large hard tablets to make slugs, screening of slugs, mixing with lubricants and disintegrating agents and finally tablet compression. In the direct compression method, the milled ingredients are mixed and then merely tableted by compression.

The present invention is further illustrated by the following Examples. These Examples are not to be regarded as limiting the scope of the invention or the manner in which it may be practiced.

### Examples

### Example 1

The following example details testing of potential active compositions for treatment of solutions with characteristic garlic aroma.

In Vitro Dose Response:

A model solution containing raw chopped garlic (20 g) dispersed in water (200 ml) was prepared by blending in a Waring commercial blender (Waring Commercial, Torrington, Conn.) at low power for 30 seconds and at high power for 30 seconds. The resulting solution was homogeneous with no visible garlic particles present.

A portion of the raw, macerated garlic solution (5 g) was treated with different levels of potential ameliorating actives and the headspace was extracted and analyzed (as described in greater detail below) to determine the effect of the potential actives on the headspace concentration of compounds contributing to oral cavity malodor associated with consumption of garlic. Two actives (menthone and caryophyllene) were tested for such headspace concentration reduction efficacy.

The amounts of actives to be tested were initially determined utilizing bench level sensory screening (i.e., sniffing the odor intensity of a sample of the macerated garlic solution before and after introduction of various amounts of active to determine its effect). Once a level is determined to provide a reduction in odor, greater and lesser amounts are tested in further detail as set forth below.

Menthone was tested at the following levels: 3 mg/g raw garlic solution, 1 mg/g raw garlic solution and 0.3 mg/g raw garlic solution. Menthone utilized in testing was produced by I.P. Callison and Sons, Lot #0525015 (I.P. Callison & Sons 2400 Callison Road NE Lacey, Wash. 98516).

Caryophyllene was tested at the following levels: 0.4 mg/g raw garlic solution, 0.24 mg/g raw garlic solution and 0.1 mg/g raw garlic solution. β-caryophyllene was obtained from Citrus and Allied Essences LTD, Lot #42611 (Citrus & Allied Essences, Ltd. 3000 Marcus Avenue Lake Success, N.Y. 11042 (718) 343-0030).

Headspace of a vessel containing a portion of the garlic solution (5 ml) treated with a potential active was extracted for 60 minutes utilizing solid phase microextraction (SPME) with a 75 μm Carboxen-polydimethyl siloxane fiber (Supelco, Bellefonte, Pa.).

The SPME fiber assembly was injected into an Agilent 6890 gas chromatograph (GC)/mass spectrometer (MS) modified for multidimensional analyses and equipped with a sniff port and Aroma Trax software (Microanalytics, Round Rock, Tex.). Fibers remained in the GC injection port for five minutes following injection.

The GC/MS operating parameters included a He carrier gas flow rate of 6.5 ml/min, split mode (2:1) and the injector set at 250° C. Column 1 was 12 meter, 0.53 mm I.D. with a methyl silica stationary phase. Column 2 was a 25 meter, 0.53 mm DB-5 capillary column. The oven was programmed to hold at 40° C. for 3 minutes and to heat to 220° C. at a rate of 7° C./min and hold at 220° C. for 20 minutes. The MS operated in Electron Impact Mode (E.I.) at 70 eV.

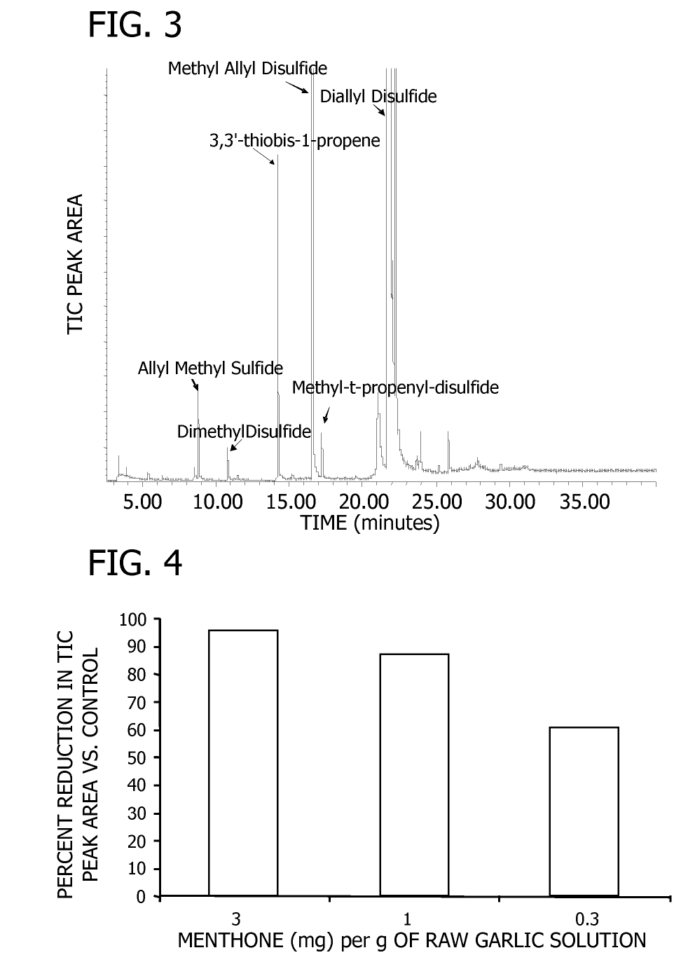

The results of GC/MS analysis of the control raw garlic solution are shown in FIG. 3 (a total ion current (TIC) chromatogram of a sample of the headspace of the control solution).

Reductions in headspace concentration of diallyl disulfide observed with addition of menthone to a raw garlic solution at varying levels based on comparison with the GC/MS results of the control garlic solution are shown in FIG. 4; Table 1 provides the analytical dose response data. As shown in FIG. 4, menthone reduced the diallyl disulfide headspace concentration of the solution of macerated garlic from 61% (at an active concentration of 0.3 mg/g solution) to 96% (at an active concentration of 3 mg/g solution).

TABLE 1
Menthone (mg menthone per g garlic solution) reduction of diallyl
disulfide headspace concentration: analytical dose response data.

| | GC/MS Peak Area | | | |
|---|---|---|---|---|
| | Control | 0.3 mg/g | 1 mg/g | 3 mg/g |
| Run 1 | 154604684 | 58241626 | 16829160 | 4757863 |
| Run 2 | 132204464 | 60764744 | 16827232 | 5868573 |
| Run 3 | 139689604 | 46610414 | 20835556 | 6746448 |
| Mean | 142166251 | 55205595 | 18163983 | 5790961.3 |
| Standard | 11403631 | 7549793.3 | 2313650.6 | 996561.72 |
| Deviation | | | | |
| Standard | 6583889.4 | 4358875.2 | 1335786.8 | 575365.17 |
| Error | | | | |
| % RSD | 8.02 | 13.67 | 12.73 | 17.20 |

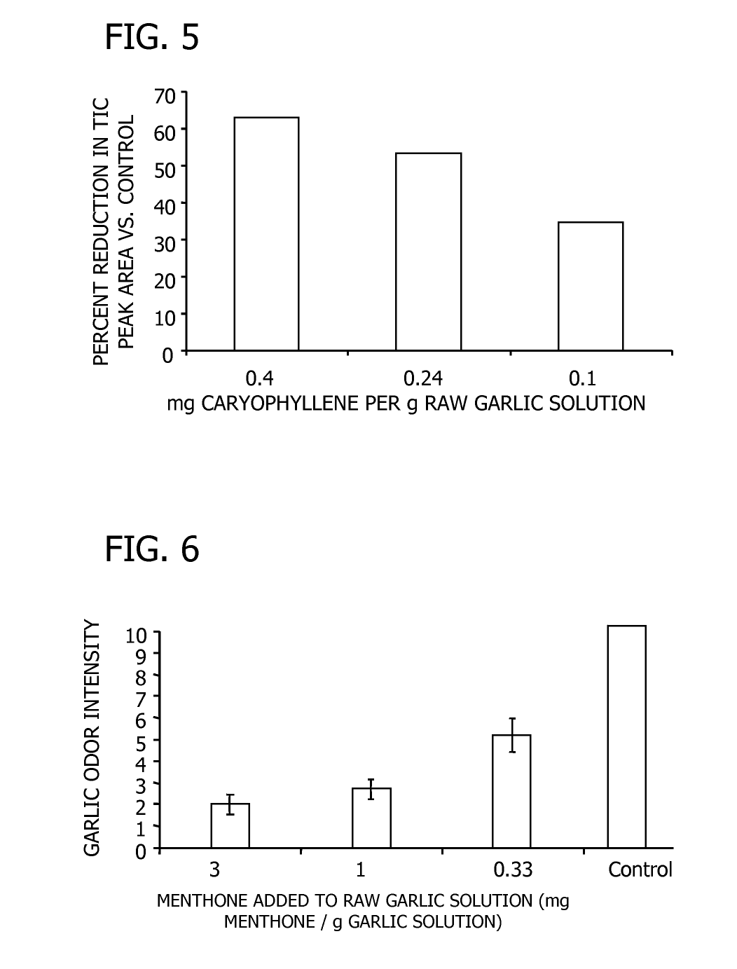

Reductions in headspace concentration of diallyl disulfide observed with addition of caryophyllene at varying levels to a raw garlic solution based on comparison with the GC/MS results of the control garlic solution are shown in FIG. 5; Table 2 provides the analytical dose response data. Active levels added were less than used with menthone due to the often-perceived woody flavor of caryophyllene. As shown in FIG. 5, diallyl disulfide headspace levels were decreased from 34% (at an active concentration of 0.1 mg/g) to 63% (at an active concentration of 0.4 mg/g).

TABLE 2
Caryophyllene (mg caryophyllene per g garlic solution)
reduction of diallyl disulfide headspace concentration:
analytical dose response data.

| | GC/MS Peak Area | | | |
|---|---|---|---|---|
| | Control | 0.4 mg/g | 0.24 mg/g | 0.1 mg/g |
| Run 1 | 1.55 × 10⁸ | 91167385 | 69400527 | 49803124 |
| Run 2 | 1.32 × 10⁸ | 94368797 | 61729644 | 56316479 |
| Run 3 | 1.4 × 10⁸ | 93422075 | 67591321 | 52155883 |
| Mean | 1.42 × 10⁸ | 92986086 | 66240497 | 52758495 |
| Standard | 11403631 | 1644635 | 4009882 | 3298227 |
| Deviation | | | | |
| Standard | 6583889 | 949530.5 | 2315107 | 1904233 |
| Error | | | | |
| % RSD | 8.02 | 1.76 | 6.05 | 6.25 |

Comparison of the actives indicates approximate parity between menthone and caryophyllene.

Sensory Analysis:

Sensory analysis of the solutions utilized for analytical analyses of the raw garlic described above was conducted by ten panelists who evaluated the odor intensity of the model solution (raw macerated garlic) with and without the different levels of added active. Odor intensity was assessed utilizing a 0-100 point scale ballot with 0=no odor and 100=very strong odor.

The results for menthone are shown in FIG. 6 and Table 3.

TABLE 3
Panelist responses (N = 10) in rating aroma intensities of garlic
odor solution treated with different concentrations of menthone.

| | | Odor Intensity (0-100) | | |
|---|---|---|---|---|
| | Panelist | 3 mg/g | 1 mg/g | 0.33 mg/g |
| 1 | LD | 4 | 2 | 8 |
| 2 | DB | 1 | 2 | 3 |
| 3 | AC | 1 | 4 | 7 |
| 4 | ED | 2 | 1 | 1 |
| 5 | RB | 0 | 2 | 1 |
| 6 | DW | 4 | 2 | 6 |
| 7 | KF | 2 | 4 | 7 |
| 8 | AA | 3 | 6 | 7 |
| 9 | SM | 0 | 2 | 7 |
| 10 | RD | 3 | 2 | 5 |
| | Mean | 2 | 2.7 | 5.2 |
| | Standard | 1.4 | 1.4 | 2.4 |
| | Deviation | | | |
| | Standard | 0.44 | 0.44 | 0.78 |
| | Error | | | |

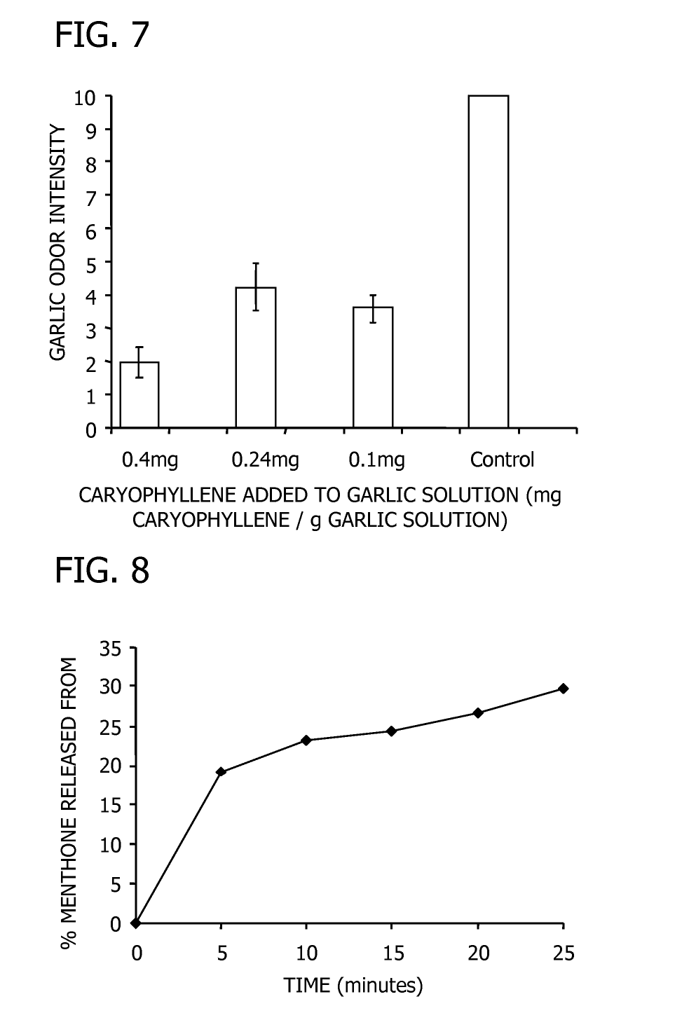

The results for caryophyllene are shown in FIG. 7 and Table 4.

TABLE 4
Panelist responses (N = 10) in rating aroma intensities of garlic
odor solution treated with different concentrations of caryophyllene.

| | | Odor Intensity (0-100) | | |
|---|---|---|---|---|
| | Panelist | 0.4 mg/g | 0.2 mg/g | 0.1 mg/g |
| 1 | KF | 3 | 5 | 7 |
| 2 | RB | 1 | 3 | 4 |
| 3 | MT | 2 | 4 | 5 |
| 4 | DB | 5 | 5 | 6 |
| 5 | AC | 3 | 4 | 2 |
| 6 | SM | 4 | 9 | 5 |
| 7 | DC | 4 | 8 | 4 |
| 8 | HV | 3 | 9 | 6 |
| 9 | RB | 4 | 5 | 5 |
| 10 | MH | 0 | 3 | 4 |
| | Mean | 2.9 | 5.5 | 4.8 |
| | Standard | 1.4 | 2.2 | 1.3 |
| | Deviation | | | |
| | Standard | 0.4 | 0.6 | 0.4 |
| | Error | | | |

The sensory dose response data indicated a trend similar to that exhibited by the analytical, in vitro analysis. As shown in FIG. 6, menthone reduced the odor of raw macerated garlic in water between 48% (at an active concentration of 0.3 mg/g) to 80% (at an active concentration of 3 mg/g). As shown in FIG. 7, caryophyllene addition decreased odor intensities between 52% (at an active concentration of 0.1 mg/g) to 71% (at an active concentration of 0.4 mg/g). Generally, caryophyllene was more effective considering the low concentration of active utilized which provided odor reduction of 71%.

### Example 2

This example details active component release (i.e., menthone and caryophyllene) from a standard chewing gum base (utilized in Peppermint Eclipse®, which is commercially available from the Wm. Wrigley Jr. Company (Chicago, Ill.)). Menthone and caryophyllene were added to the gum coating. In order for an active compound to be suitable for use with chewing gum as a delivery vehicle, it typically releases from the gum matrix and into the oral cavity during chewing so that it may interact with garlic odorants present in the oral cavity.

The method to measure active release and rate of release involved having 5 panelists chew one serving of gum each (2 pellets, 1.5 g each), for each of the following times: 5, 10, 20, 25 minutes. After chewing for each time period, gum cuds were collected (a minimum of 6 cuds) and dissolved in a chloroform solvent with undecane as the internal standard. The solution was shaken for six hours to ensure salvation. Liquid was then removed and purified with solid phase extraction (SPE) utilizing a Millipore (Billerica, Mass.) Millex-FH hydrophobic poytetrafluoroethylene (PTFE) membrane having a pore size of 0.45 μm. For non-volatile active components, the aqueous layer was removed and analyzed by high performance liquid chromatography (HPLC).

The amount of active in the extracted liquid phase was used to determine the amount of active released from the gum during chewing. Undecane was utilized as an internal standard and a calibration curve constructed. The chloroform layer (bottom layer) was removed by Pasteur pipette and placed in a GC vial and capped with a crimped cap and Teflon septa. The liquid was injected into the GC (described above in Example 1) for active component quantification utilizing an Agilent 7683 Series Autosampler (Agilent, Palo Alto, Calif.) set to inject 5 μl. Injections are done in triplicate to ensure accuracy and precision.

The active amounts remaining in the gum cuds containing each of the potential actives were compared to a control gum containing the active which was not chewed.

As shown in Table 5, the total amount of menthone in the 2 pellets of control gum (i.e., Peppermint Eclipse®), without additional menthone, was 10.7 mg.

TABLE 5
Menthone in control chewing gum (total in 2 pellets).

| Time | % present | |
|---|---|---|
| (min) | in sample | Menthone (mg) |
| 0 | 0.357 | 10.7 mg |

When testing menthone as an active, the initial amount was 14.2 mg menthone (i.e., an additional 3.5 mg of menthone).

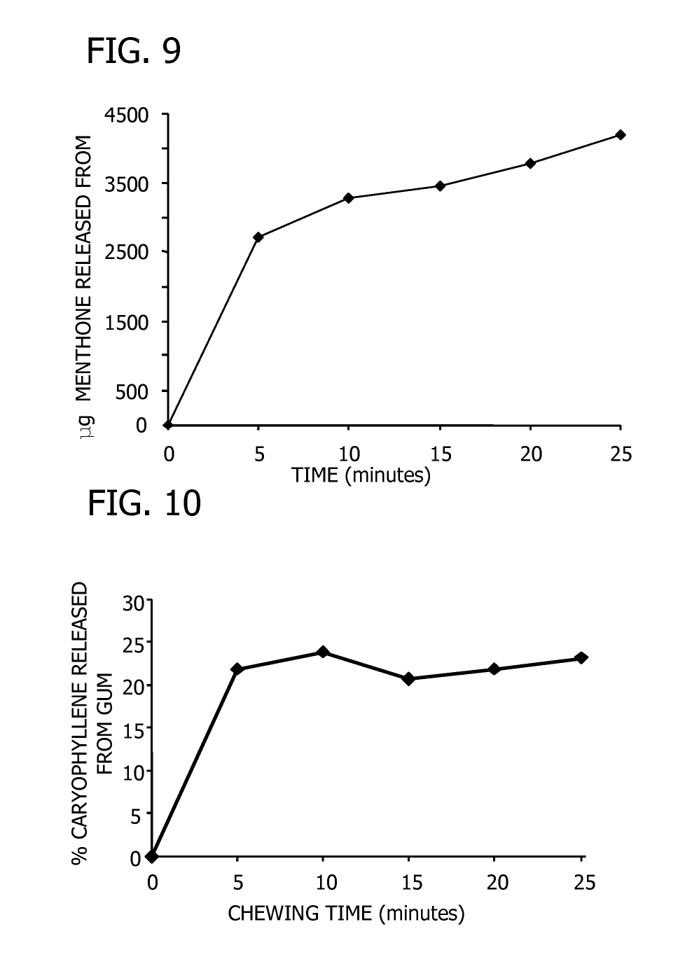

The amount of menthone remaining in the gum after 5, 10, 15, 20 and 25 minutes of chewing was determined. For example, 9.81 mg remained after 25 minutes of chewing. Thus, thirty-one percent (4.39 mg) of menthone were released during 25 minutes of chewing. FIGS. 8 and 9 show the rate of menthone release during 25 minutes of chewing in terms of the percentage of menthone released from the gum and amount (μg) of menthone released from the gum, respectively.

As shown in Table 6, the total amount of caryophyllene in the 2 pellets of the control gum, without additional caryophyllene, was 1.35 mg.

TABLE 6
Caryophyllene in control chewing gum (total in 2 pellets).

| Time | % present | caryophyllene |
|---|---|---|
| (min) | in sample | (mg) |
| 0 | 0.023 | 1.35 mg |

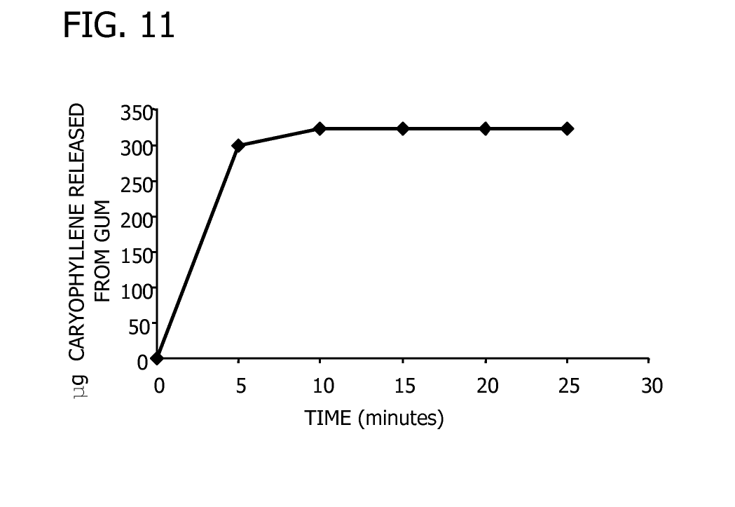

The amount of caryophyllene remaining in the gum after 5, 10, 15, 20 and 25 minutes of chewing was determined. For example, 1.04 mg remained after 25 minutes of chewing. Thus, 23% of the caryophyllene was released during the 25 minutes of chewing. FIGS. 10 and 11 show the rate of caryophyllene release during 25 minutes of chewing in terms of the percentage of caryophyllene released from the gum and amount (μg) of caryophyllene released from the gum, respectively.

### Example 3

The following example details testing of menthone for treatment of solutions with characteristic garlic aroma.

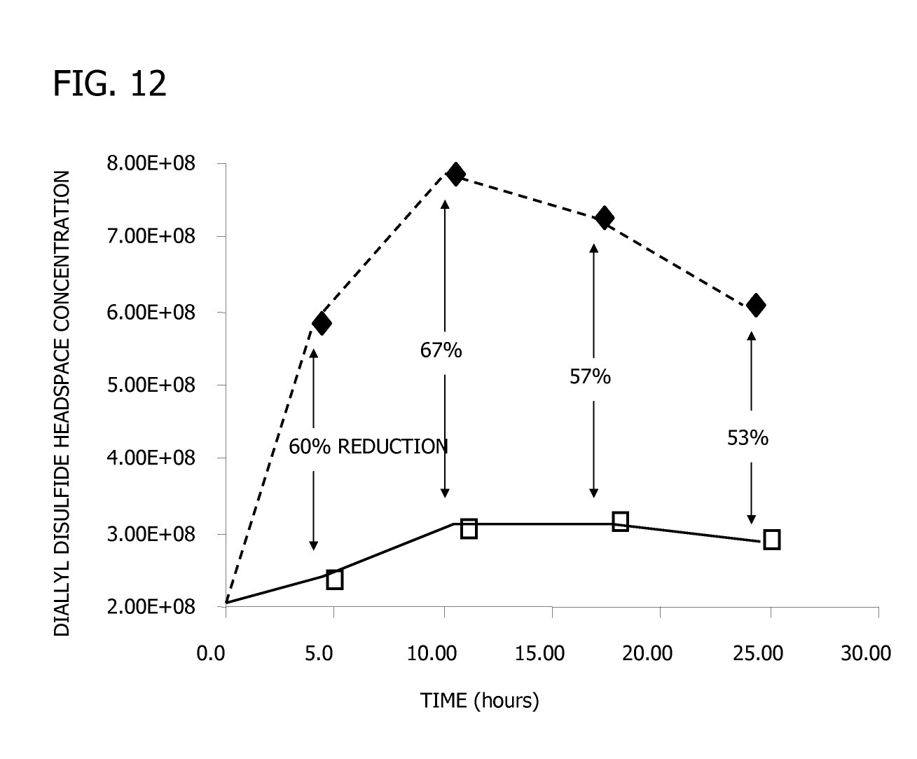

A model solution containing raw chopped garlic dispersed in water was prepared as described in Example 1. Menthone (1.63 mg) was introduced to a portion of the model solution (2.5 g) and a portion of the vessel headspace was extracted and its diallyl disulfide content was analyzed at 5 hour intervals using GC/MS analysis as described in Example 1. For comparison purposes, the diallyl disulfide content of the headspace of a control model solution (i.e., no menthone added) was also analyzed. The results are shown in FIG. 12; as shown, the headspace of the vessel containing the model solution treated with menthone contained at least 50% less diallyl disulfide at each headspace analysis interval.

### Example 4

This example details testing of active composition at varying concentrations for treatment of solutions with characteristic garlic aroma.

Model solutions containing raw chopped garlic dispersed in water were prepared as described in Example 1. Portions of model solution (2.5 g) were treated with menthone, menthone and caryophyllene, or caryophyllene in the following amounts:
(1) 0.5 mg menthone (0.2 mg/g solution)
(2) 1 mg menthone (0.4 mg/g solution)
(3) 1.5 mg menthone (0.6 mg/g solution)
(4) 2 mg menthone (0.8 mg/g solution)
(5) 0.25 mg menthone (0.1 mg/g solution)+0.25 mg (0.1 mg/g solution) caryophyllene
(6) 0.5 mg menthone (0.2 mg/g solution)+0.5 mg (0.2 mg/g solution) caryophyllene
(7) 0.75 mg menthone (0.3 mg/g solution)+0.75 mg (0.3 mg/g solution) caryophyllene
(8) 0.75 mg caryophyllene (0.3 mg/g solution)
(9) 1.5 mg caryophyllene (0.6 mg/g solution)

A portion of the headspace of the vessel containing each treated solution was extracted and its diallyl disulfide content was analyzed as described in Example 1. For comparison purposes, the diallyl disulfide content of the headspace of vessels containing control model solutions (i.e., no active added) were also analyzed.

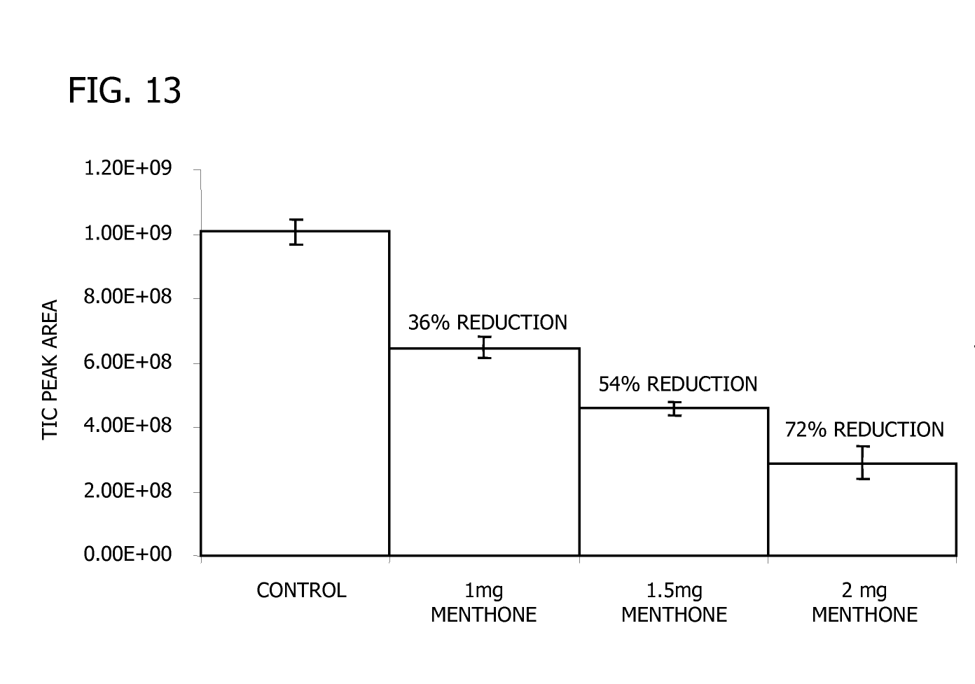

FIG. 13 shows the peak area of total ion current (TIC) chromatograms for samples (2)-(4) and a control sample obtained by GC/MS analysis as described in Example 1.

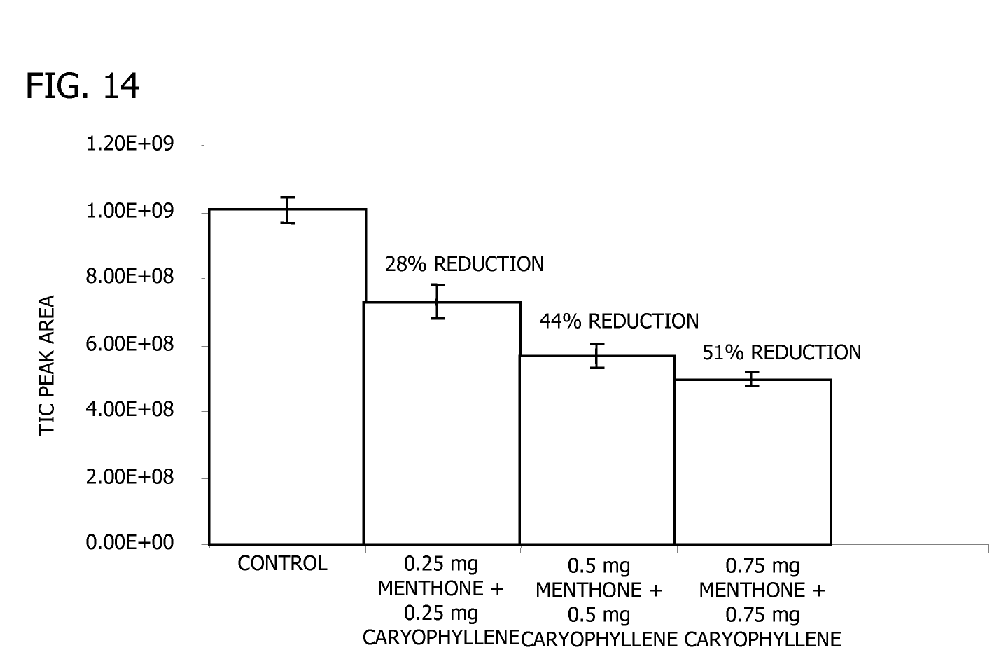

FIG. 14 shows the peak area of total ion current (TIC) chromatograms for samples (5)-(7) and a control sample obtained by GC/MS analysis as described in Example 1.

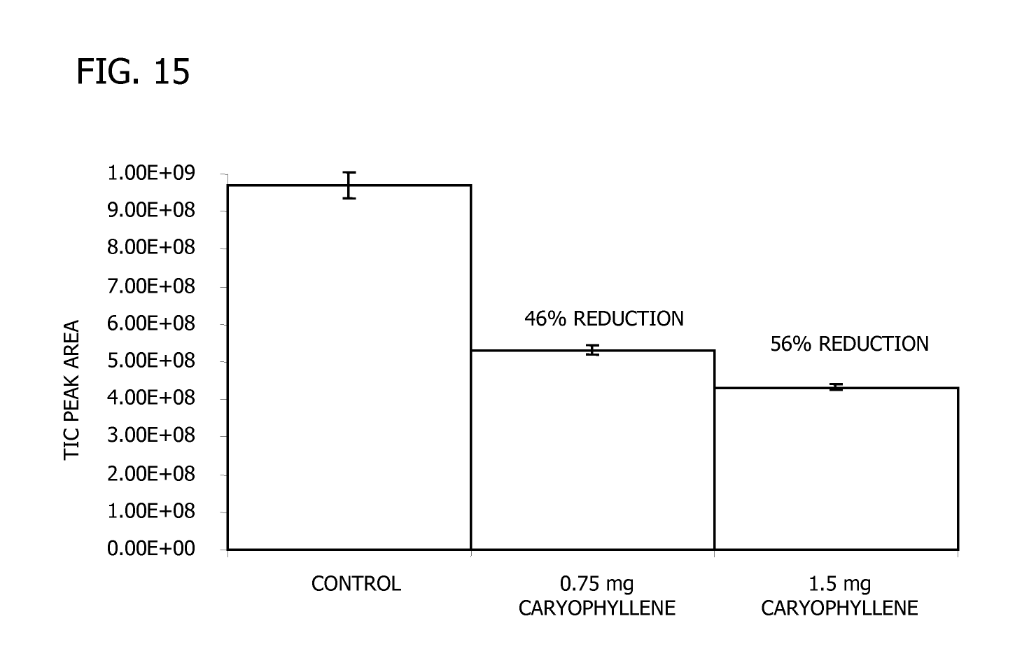

FIG. 15 shows the peak area of total ion current chromatograms for samples (8) and (9) and a control sample obtained by GC/MS analysis as described in Example 1.

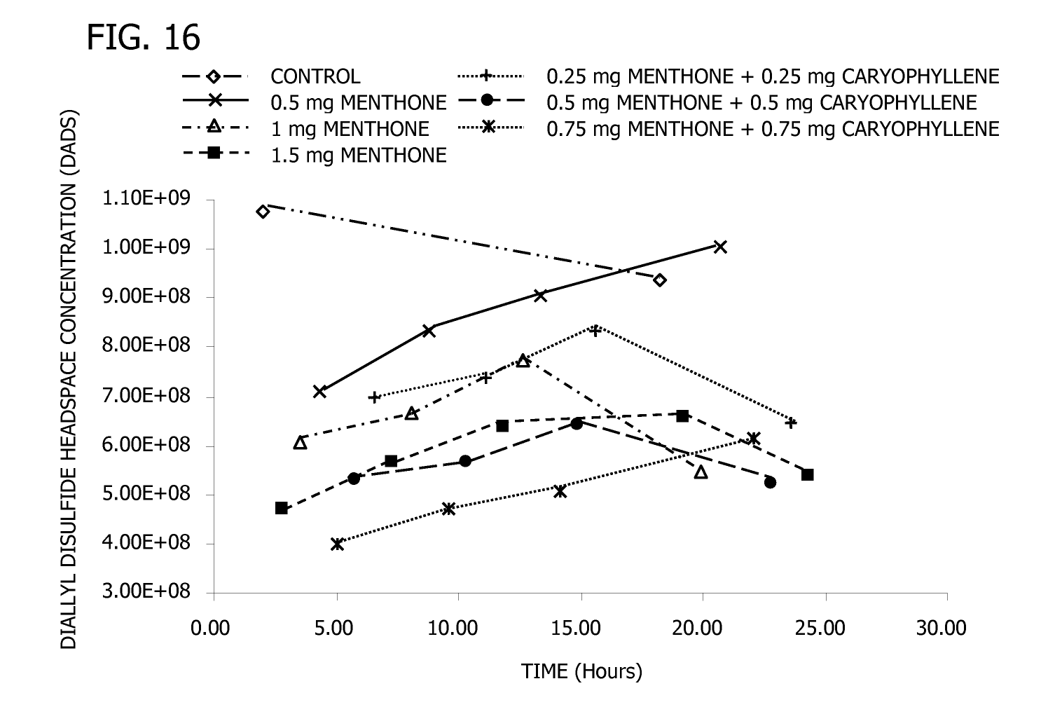

FIG. 16 shows the diallyl disulfide concentration determined by GC/MC analysis at 5 hour intervals for a control sample and samples (1)-(3) and (5)-(7).

### Examples 5-16

Typical gum formulations are set forth in Tables 7 and 8.

TABLE 7
Chewing Gum Examples (% by weight)

| | | | | | | Example |
|---|---|---|---|---|---|---|
| Ingredient | Example 5 | Example 6 | Example 7 | Example 8 | Example 9 | 10 |
| Base | 30.80 | 31.90 | 30.50 | 35.50 | 36.50 | 30.00 |
| Sorbitol | 45.55 | 41.55 | 48.45 | 41.44 | 40.94 | 46.00 |
| Talc | 15.00 | 13.00 | 14.00 | 9.00 | 13.00 | 15.00 |
| Color | 0.05 | 0.05 | 0.05 | 0.05 | 0.05 | 0.05 |
| Glycerin | 2.50 | | 2.57 | 6.00 | 1.00 | 2.10 |
| Water | 1.00 | 1.50 | | 1.50 | 1.50 | 0.95 |
| Flavor | 3.00 | 3.00 | 0.86 | 3.00 | 5.00 | 2.00 |
| Acid | | | | | | 0.60 |
| Artificial Sweetener | 0.60 | 3.00 | 2.00 | 3.00 | 1.00 | 1.50 |
| Menthol | 0.50 | | 0.50 | 0.50 | 0.50 | 1.00 |
| Cooling Agent | 1.00 | 1.00 | 1.00 | | 0.50 | |
| 80/20 (wt/wt) mixture | | | 0.07 | | | 0.80 |
| menthone/isomenthone* | | | | | | |
| 70/30 (wt/wt) mixture | | 2.50 | | 0.001 | 0.01 | |
| menthone/isomenthone | | | | | | |
| Caryophyllene | | 2.50 | | 0.001 | | |
| (80% pure) | | | | | | |
| TOTAL: | 100.00% | 100.00% | 100.00% | 100.00% | 100.00% | 100.00% |

*(e.g., Menthone/Isomenthone mixture available from I.P Callison & Sons, 98% Pure)

TABLE 8
Chewing Gum Examples (% by weight)

| | Example | Example | Example | Example | Example | Example |
|---|---|---|---|---|---|---|
| Ingredient | 11 | 12 | 13 | 14 | 15 | 16 |
| Base | 25.65 | 33.00 | 36.34 | 34.50 | 34.50 | 32.00 |
| Sugar | 45.00 | 53.95 | | 41.45 | | |
| Maltitol | | | 48.00 | | 2.50 | 46.00 |
| Xylitol | | | | | 46.00 | |
| Water | | | | | 1.05 | 0.80 |
| Talc | 15.00 | | 4.00 | 10.00 | 1.19 | 13.92 |
| Color | 0.05 | 0.05 | 0.25 | 0.05 | 0.01 | |
| Corn Syrup | 8.00 | 6.25 | | 3.95 | | |
| Sorbitol Syrup | | | 7.10 | | 4.50 | 2.50 |
| Flavor | 1.50 | 3.00 | 2.50 | 3.00 | 5.00 | 2.80 |
| Acid | 1.00 | | | 4.00 | | |
| Artificial Sweetener | 3.00 | 2.00 | 0.85 | 1.00 | 3.00 | 1.90 |
| Menthol | | 0.50 | 0.50 | | 0.75 | |
| Cooling Agent | | 1.00 | 0.25 | 0.05 | 1.00 | |
| 90/10 (wt/wt) | 0.80 | | 0.07 | | 0.25 | 0.08 |
| mixture | | | | | | |
| menthone/isomenthone | | | | | | |
| 75/25 (wt/wt) | | 0.10 | 0.07 | 1.00 | | |
| mixture | | | | | | |
| menthone/isomenthone | | | | | | |
| Neat caryophyllene | | 0.15 | 0.07 | 1.00 | 0.25 | |
| TOTAL: | 100.00% | 100.00% | 100.00% | 100.00% | 100.00% | 100.00% |

### Example 17

Menthone and β-caryophyllene are premixed together with a suitable flavoring agent and added to the gum composition of Example 5 to provide a menthone content of approximately 10% and a β-caryophyllene content of approximately 4%, by weight of the gum composition.

### Example 18

A mixture of 80 parts menthone and 20 parts isomenthone is premixed with a suitable flavoring agent and added to the gum composition of Example 5 to provide a menthone content of approximately 0.15%, by weight of the gum composition.

### Example 19

98% pure menthone and 98% β-caryophyllene are premixed together with a suitable flavoring agent and added to the gum composition of Example 5 to provide a menthone content of approximately 0.15% and a β-caryophyllene content of approximately 0.32%, by weight of the gum composition.

### Example 20

A mixture of menthone and β-caryophyllene are premixed together with a suitable flavoring agent and added to the gum composition of Example 5 to provide a menthone content of approximately 2.50% and a β-caryophyllene content of approximately 5.00%, by weight of the gum composition.

### Examples 21-26

Typical coating formulations that may be incorporated into chewing gums and confections of the present invention are set forth in Table 9. The coatings may be applied to the chewing gum or confection using any of various methods known in the art including, for example, pan coating (see, for example, U.S. Pat. No. 4,753,790 to Silva et al), fluid bed coating (see, for example, U.S. Pat. No. 5,641,536 to Lech, et al.), coextrustion, dip coating, and/or film coating (see, for example, U.S. Pat. No. 4,317,838 to Cherukuri et al.). For example, the coating solutions may be pan coated onto a sugar free chewing gum composition, fluid bed coated onto a chewy candy composition, concentrically coextruded with a flavor oil to encapsulate the flavor oil, used for dip-coating dry fruits, pan coated onto a hard-boiled candy with a liquid center, film coated onto a pharmaceutical capsule containing an active ingredient, and/or used in coating a nutraceutical tablet containing garlic.

TABLE 9
Coating Solution Examples (% by weight)

| | Example | Example | Example | Example | Example | Example | Example |
|---|---|---|---|---|---|---|---|
| Ingredient | 21 | 21 (a) | 22 | 23 | 24 | 25 | 26 |
| Maltitol | | | | 70.9 | | | 25.15 |
| Isomalt | 89.4 | 89.0 | | | | 75.00 | |
| Xylitol | | | | | 97.94 | | |
| Erythritol | | | | | | 10.00 | 25.00 |
| Sugar | | | 80.0 | | | | 22.00 |
| Dextrose | | | | | | 2.00 | |
| Gum Acacia | 5.00 | 5.00 | | | 1.00 | | |
| Gelatin | | | | 10.00 | | | |
| Starch | | | | | | 2.00 | |
| Modified Starch | | | 6.00 | | | 3.00 | 18.00 |
| Color | | | | 1.00 | 0.05 | 0.85 | |
| Titanium Dioxide | 1.00 | 1.00 | 1.00 | | | | |
| Wax | | | 0.02 | 0.15 | | 0.30 | |
| Flavor | 0.50 | 0.50 | 1.00 | 0.85 | | | |
| Acid | | | | | | 0.55 | 0.85 |
| Artificial | 0.75 | 0.71 | | 0.55 | 0.08 | | 1.00 |
| Sweetener | | | | | | | |
| Menthol | 0.30 | 0.30 | | | 0.88 | 0.05 | |
| Cooling Agent | 0.05 | 0.05 | | 0.55 | | 0.15 | |
| Neat Magnolia | | 0.08 | | | | | |
| Bark Extract | | | | | | | |
| 95/5 | | | | 22.00 | | | |
| (wt/wt) menthone/isomenthone | | | | | | | |
| mixture | | | | | | | |
| 80/20 | 4.80 | 4.80 | 11.98 | | 10.00 | 6.10 | |
| (wt/wt) menthone/isomenthone | | | | | | | |
| mixture* | | | | | | | |
| Neat | 8.20 | 8.20 | | 3.00 | 5.00 | 0.05 | 15.00 |
| caryophyllene | | | | | | | |
| TOTAL: | 100.00% | 100.00% | 100.00% | 100.00% | 100.00% | 100.00% | 100.00% |

*(e.g., Menthone/Isomenthone mixture available from I.P Callison & Sons, 98% Pure)

### Example 27

The composition of Example 21 is pan coated onto the gum composition of Example 5 to provide a menthone content of approximately 1.4% and a β-caryophyllene content of approximately 2.50%, by weight of the total gum composition.

### Example 28

The composition of Example 22 is pan coated onto the gum composition of Example 6 to provide a menthone content of approximately 6.0% and a β-caryophyllene content of approximately 2.50%, by weight of the total composition.

### Example 29

The composition of Example 24 is pan coated onto the candy composition as provided in Table 12.

### Examples 30-34(a)

Typical hard candy formulations are set forth in Tables 10 and 10(a).

TABLE 10
Breath Freshening Candy Formulations (% by weight)

| | | | | | Exam- |
|---|---|---|---|---|---|
| | Example | Example | Example | Example | ple |
| Ingredient | 30 | 31 | 32 | 33 | 34 |
| Corn Syrup | 45.00 | 42.00 | | | 47.00 |
| Sugar | 53.24 | 49.00 | | | 47.00 |
| Polyalcohols | | | 95.00 | 93.50 | |
| Flavor | 1.00 | 5.00 | 3.00 | 2.00 | 2.50 |
| Color | 0.50 | 1.00 | 0.60 | 0.80 | 0.50 |
| Menthol | 0.25 | | | 0.50 | |
| 95/5 (wt/wt) | 0.01 | 0.05 | 1.20 | 1.50 | |
| menthone/ | | | | | |
| isomenthone | | | | | |
| mixture | | | | | |
| 90/10 | | 0.95 | | | 3.00 |
| (wt/wt) | | | | | |
| menthone/ | | | | | |
| isomenthone | | | | | |
| mixture | | | | | |
| Neat | | 2.00 | | 1.50 | |
| Caryophyllene | | | | | |
| High | | | 0.20 | 0.20 | |
| Intensity | | | | | |
| Sweetener | | | | | |
| Total | 100.00 | 100.00 | 100.00 | 100.00 | 100.00 |

TABLE 10 (a)
Breath Freshening Candy Formulations (% by weight)

| | | | | | Exam- |
|---|---|---|---|---|---|
| | Example | Example | Example | Example | ple |
| Ingredient | 30 (a) | 31 (a) | 32 (a) | 33 (a) | 34 (a) |
| Corn Syrup | 45.00 | 42.00 | | | 47.00 |
| Sugar | 53.24 | 49.00 | | | 47.00 |
| Polyalcohols | | | 95.00 | 93.50 | |
| Flavor | 1.00 | 5.00 | 3.00 | 2.00 | 2.50 |
| Color | 0.50 | 1.00 | 0.60 | 0.80 | 0.50 |
| Menthol | 0.25 | | | 0.50 | |
| 90/10 | 0.01 | 0.05 | 1.20 | 1.50 | |
| (wt/wt) | | | | | |
| menthone/ | | | | | |
| isomenthone | | | | | |
| mixture | | | | | |
| 80/20 (wt/wt) | | 0.95 | | | 3.00 |
| menthone/ | | | | | |
| isomenthone | | | | | |
| mixture | | | | | |
| Neat | | 2.00 | | 1.50 | |
| Caryophyllene | | | | | |
| High | | | 0.20 | 0.20 | |
| Intensity | | | | | |
| Sweetener | | | | | |
| Total | 100.00 | 100.00 | 100.00 | 100.00 | 100.00 |

### Example 35

A typical hard candy formulation is set forth in Table 11.

TABLE 11
Breath Freshening Candy Formulations (% by weight)

| Compound | Amount |
|---|---|
| Sorbitol | 94-98% |
| Magnesium Stearate | 0.6-0.8% |
| Colorants | 1.5-4.0% |
| Flavor | <1.0% |
| Sweetener | 0.1-0.2% |
| Menthol | 0.0-4% |
| 95/5 (wt/wt) | 0.0-4% |
| menthone/isomenthone | |
| mixture | |
| 70/30 (wt/wt) | 0.0-4% |
| menthone/isomenthone | |
| mixture | |
| Neat Magnolia Bark | 0.0-4% |
| Extract | |
| Neat caryophyllene | 0.0-4% |

### Example 36

A typical pressed mint formulation is set forth in Table 12.

TABLE 12
Breath Freshening Candy Formulations (% by weight)

| Compound | Amount |
|---|---|
| Mint granulation | 97% |
| Ca(OH)₂ | 0.5% |
| Calcium stearate | 1% |
| Flavoring | 0.4% |
| Menthol | 0.0-4% |
| 50/50 | 0.0-4% |
| menthone/isomenthone | |
| mixture | |
| 70/30 | 0.0-4% |
| Menthone/isomenthone | |
| mixture | |
| Neat Magnolia Bark | 0.0-4% |
| Extract | |
| Neat caryophyllene | 0.0-4% |

### Example 37

A typical boiled drop formulation is set forth in Table 13.

TABLE 13
Breath Freshening Candy Formulations (% by weight)

| Compound | Amount |
|---|---|
| Sugar | 73.3% |
| Corn Syrup | 25.0% |
| Ca(OH)₂ | 0.2% |
| Flavoring | 0.5% |
| Menthol | 0.0-2.5% |
| 60/40 (wt/wt) | 0.0-2.5% |
| menthone/isomenthone | |
| mixture | |
| 85/15 (wt/wt) | 0.0-2.5% |
| Menthone/isomenthone | |
| mixture | |
| Neat Magnolia Bark | 0.0-2.5% |
| Extract | |
| Neat caryophyllene | 0.0-2.5% |

### Examples 38-42

Typical pressed mint formulations are set forth in Table 14. These formulas may be utilized in mints in a variety of shapes and sizes, including mints that are multi-layered, coated, and/or designed to have unique textural properties.

TABLE 14
Breath Freshening Compressed Mint Formulations (% by weight)

| | Example | Example | Example | Example | Example | Example |
|---|---|---|---|---|---|---|
| Ingredient | 38 | 38 (a) | 39 | 40 | 41 | 42 |
| Sorbitol | 97.63 | 97.60 | 97.43 | 96.83 | 95.83 | 93.83 |
| Flavor | 1.00 | 1.00 | 1.00 | 1.00 | 1.00 | 1.00 |
| Mg stearate | 0.97 | 0.97 | 0.97 | 0.97 | 0.97 | 0.97 |
| High Intensity | 0.20 | 0.20 | 0.20 | 0.20 | 0.20 | 0.20 |
| Sweetener | | | | | | |
| Menthol | | | | | | 0.25 |
| 80/20 (wt/wt) | 0.10 | 0.10 | 0.20 | 0.50 | 1.00 | 1.75 |
| Menthone/isomenthone | | | | | | |
| mixture* | | | | | | |
| Magnolia Bark | | 0.03 | | | | |
| Extract | | | | | | |
| Neat caryophyllene | 0.10 | 0.10 | | 0.50 | 1.00 | |
| Total | 100.00 | 100.00 | 100.00 | 100.00 | 100.00 | 100.00 |

*(e.g., Menthone/Isomenthone mixture available from I.P Callison & Sons, 98% Pure)

### Examples 43-46

Typical coating formulations are set forth in Table 15. Any of the below coating formulations may be employed with the compositions of any of the previous examples, or with compositions that do not contain menthone, isomenthone, and/or caryophyllene.

TABLE 15

| | Example | Example | Example | Example |
|---|---|---|---|---|
| Ingredient | 43 | 44 | 45 | 46 |
| Sorbitol | 98.20 | 43.26 | 98.05 | 1.00 |
| Xylitol | | 55.0 | | 97.00 |
| Titanium Dioxide | 1.00 | | 1.00 | 0.50 |
| High Intensity | 0.80 | 0.50 | 0.80 | 0.50 |
| Sweetener | | | | |
| Color | | 1.00 | | 0.85 |
| 80/20 (wt/wt) | | 0.12 | 0.15 | 0.05 |
| Menthone/isomenthone | | | | |
| mixture* | | | | |
| Neat | | 0.12 | | 0.10 |
| caryophyllene | | | | |
| TOTAL | 100.00 | 100.00 | 100.00 | 100.00 |

*(e.g., Menthone/Isomenthone mixture available from I. P Callison & Sons, 98% Pure)

### Example 47

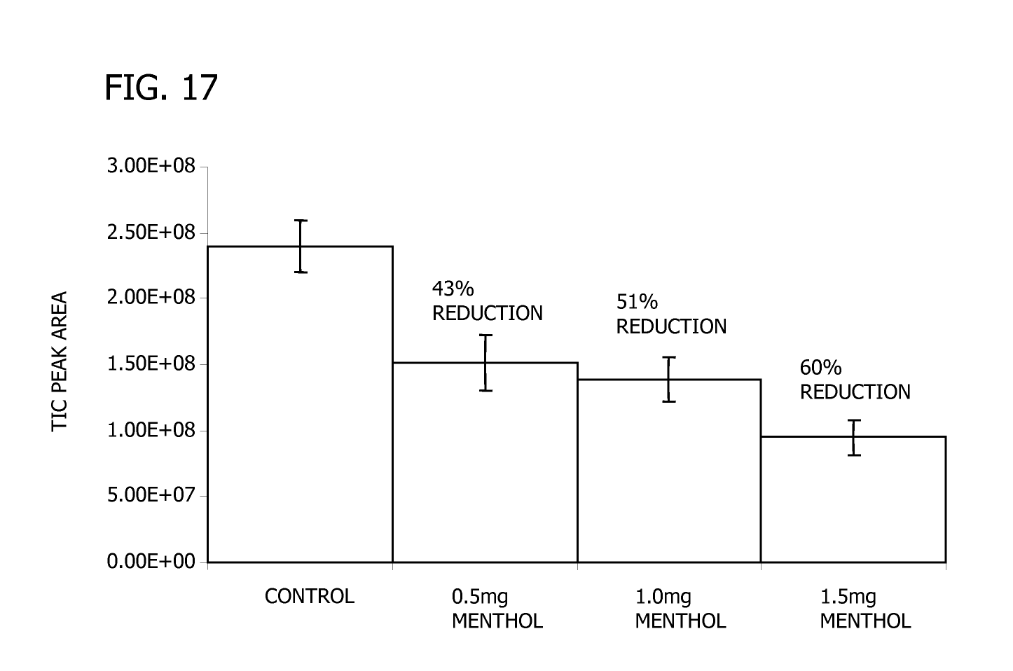

The following example details testing of menthol for treatment of solutions with characteristic garlic aroma. Model solutions containing raw chopped garlic dispersed in water were prepared as described in Example 1. Portions of model solution (2.5 g) were treated with menthol in varying amounts (0.5 mg, 1.0 mg, and 1.5 mg). As shown in FIG. 17, treatment of the model solution with 0.5 mg of menthol provided a reduction in odor-causing compounds of approximately 43%, treatment of the model solution with 1 mg of menthol provided a reduction in odor-causing compounds of approximately 51%, and treatment of the model solution with 1.5 mg of menthol provided a reduction in odor-causing compounds of approximately 60%.

### Example 48

This Example provides representative compositions for the center and coating of a pellet chewing gum prepared in accordance with the present invention, and including magnolia bark extract within the coating. The menthone/isomenthone mixture is pre-blended with the flavor prior to blending these two ingredients with the other components of the gum center.

TABLE 16
Center (% by weight)

| Ingredient | % by weight |
|---|---|
| Sorbitol | 48.20 |
| Base | 33.00 |
| Talc | 10.00 |
| Flavor | 3.50 |
| 80/20 (wt/wt) | 0.10 |
| Menthone/isomenthone | |
| mixture | |
| Glycerin | 2.20 |
| Water | 1.00 |
| Sweeteners | 2.00 |
| TOTAL | 100.00 |

TABLE 17
Coating (% by weight)

| Ingredient | % |
|---|---|
| Maltitol | 95.28 |
| Gum Arabic | 2.65 |
| Wax | 0.05 |
| Cooling Agent | 0.08 |
| Titanium Dioxide | 0.10 |
| Flavor | 1.50 |
| Sweeteners | 0.10 |
| Magnolia Bark Extract | 0.04 |
| (92% pure) | |
| TOTAL | 100.00 |

A typical serving of a coated (e.g., pelleted) chewing gum includes two pellets (approximately 1.45 grams each). The concentration of menthone in each pellet (and each serving) is approximately 0.04% by weight (i.e., approximately 1.18 mg menthone per serving), and the concentration of MBE in each pellet (and each serving) is approximately 0.03% by weight (i.e., approximately 0.79 mg MBE per serving).

### Example 49

This example details representative formulations for the center and coating of a pelleted chewing gum prepared in accordance with the present invention. During preparation, the source of menthone and flavor are pre-blended prior to blending with the other components of the center.

TABLE 18
Center (% by weight)

| Ingredient | % by weight |
|---|---|
| Sorbitol | 47.00 |
| Base | 28.70 |
| Talc | 15.00 |
| Flavor | 3.50 |
| 80/20 (wt/wt) | 0.10 |
| Menthone/isomenthone | |
| mixture | |
| Glycerin | 2.70 |
| Water | 1.00 |
| Sweeteners | 2.00 |
| TOTAL | 100.00 |

TABLE 19
Coating (% by weight)

| Ingredient | % by weight |
|---|---|
| Maltitol | 95.48 |
| Gum Arabic | 2.60 |
| Wax | 0.05 |
| Cooling Agent | 0.08 |
| Titanium Dioxide | 0.19 |
| Flavor | 1.50 |
| Sweeteners | 0.10 |
| TOTAL | 100.00 |

The present invention is not limited to the above embodiments and can be variously modified. The above description of the preferred embodiments, including the Examples, is intended only to acquaint others skilled in the art with the invention, its principles, and its practical application so that others skilled in the art may adapt and apply the invention in its numerous forms, as may be best suited to the requirements of a particular use.

With reference to the use of the word(s) comprise or comprises or comprising in this entire specification (including the claims below), unless the context requires otherwise, those words are used on the basis and clear understanding that they are to be interpreted inclusively, rather than exclusively, and applicants intend each of those words to be so interpreted in construing this entire specification.

1-11. (canceled)

12. A method for preparing an oral composition effective for reducing oral malodor, the method comprising:
introducing a source composition containing both menthone and isomenthone into a mixture of ingredients of the oral composition, wherein the total concentration of menthone and isomenthone in the source composition is at least about 60% by weight.

13. The method as set forth in claim 12 wherein the total concentration of menthone and isomenthone in the source composition is at least about 70% by weight.

14. The method as set forth in claim 12 wherein the weight ratio of menthone to isomenthone in the source composition is at least about 70:30.

15. The method as set forth in claim 14 wherein the weight ratio of menthone to isomenthone in the source composition is about 80:20.

16. The method as set forth in claim 12 wherein the oral composition is in the form of a chewing gum, confection, lozenge, pressed tablet, edible film, mouthspray, dentrifice, mouthwash, foam, toothpaste product, or a combination thereof.

17-20. (canceled)

21. The method as set forth in claim 12 further comprising introducing magnolia bark extract into the source of ingredients, wherein the magnolia bark extract is present in the oral composition at a concentration of from about 0.001% to about 10% by weight.

22. (canceled)

23. An oral composition effective for reducing oral malodor, the composition comprising a non-mint flavor and a source composition containing both menthone and isomenthone, wherein the total concentration of menthone and isomenthone in the source composition is at least about 60% by weight.

24. The oral composition of claim 23 wherein the non-mint flavor is derived from a fruit.

25. The oral composition as set forth in claim 23 wherein the total concentration of menthone and isomenthone in the source composition is at least about 70% by weight.

26. The oral composition as set forth in claim 23 wherein the weight ratio of menthone to isomenthone in the source composition is at least about 70:30.

27. The oral composition as set forth in claim 23 wherein the weight ratio of menthone to isomenthone in the source composition is about 80:20.

28. The oral composition as set forth in claim 23 wherein the concentration of menthone in the composition is from about 0.001% to about 10% by weight.

29. The oral composition as set forth in claim 23 wherein the concentration of isomenthone in the composition is from about 0.001% to about 10% by weight.

30. (canceled)

31. The oral composition as set forth in claim 23 further comprising magnolia bark extract, wherein the magnolia bark extract is present in the oral composition at a concentration of from about 0.001% to about 10% by weight.

32. An oral composition effective for reducing oral malodor, the composition comprising:
a source composition containing both menthone and isomenthone; and
magnolia bark extract.

33. The oral composition as set forth in claim 32 wherein the magnolia bark extract is present in the oral composition at a concentration of from about 0.001% to about 8% by weight.

34. The oral composition as set forth in claim 32 wherein the weight ratio of menthone to isomenthone in the source composition is at least about 70:30.

35-40. (canceled)

41. The oral composition as set forth in claim 32, wherein the composition is in the form of a coated chewing gum composition comprising a center and a coating.

42. The oral composition of claim 41 wherein menthone is present in the center and magnolia bark extract is present in the coating.

43. The oral composition of claim 42 wherein substantially all the menthone present in the oral composition is present in the center, and substantially all the magnolia bark extract present in the composition is present in the coating.

44. The oral composition of claim 32, wherein the composition is in the form of a multi-layered pressed tablet.

45. The oral composition of claim 44 wherein substantially all the menthone present in the oral composition is present in one layer, and substantially all the magnolia bark extract present in the composition is present in a different layer.
